(19) United States
(12) Patent Application Publication
(10) Pub. No.: US 2022/0401365 A1
(43) Pub. Date: Dec. 22, 2022

(54) DRY POWDER FORMULATIONS CONTAINING LEUCINE AND TRILEUCINE

(71) Applicant: AstraZeneca AB, Scdertalje (SE)

(72) Inventors: Prakash MANIKWAR, Gaithersburg, MD (US); David LECHUGA-BALLESTEROS, San Francisco, CA (US); Susan HOE, San Francisco, CA (US); Kellisa Beth HANSEN, San Francisco, CA (US); Dexter Joseph D'SA, San Francisco, CA (US); Saba GHAZVINI, Gaithersburg, MD (US)

(21) Appl. No.: 17/772,187

(22) PCT Filed: Oct. 27, 2020
(86) PCT No.: PCT/EP2020/080203

§ 371 Date: Apr. 27, 2022

Related U.S. Application Data
(60) Provisional application No. 62/926,832, filed on Oct. 28, 2019.

(51) Int. Cl. A61K 9/16; A61K 9/00; C07K 16/24

(52) U.S. Cl. CPC A61K 9/1617; A61K 9/1623; A61K 9/1694; A61K 9/0075; C07K 16/244; A61K 9/1652; C07K 2317/55

(57) ABSTRACT

The present technology relates generally to dry powder formulations comprising leucine and trileucine in specific ratios that are suitable for pulmonary delivery. Also provided are methods of preparing the dry powder formulations, and methods of administration and treatment using the dry powder formulations.

### FIELD OF THE DISCLOSURE

The present technology relates generally to dry powder formulations suitable for pulmonary delivery. Also provided are methods of preparing the dry powder formulations, and methods of administration and treatment using the dry powder formulations.

### BACKGROUND

The advantages of pulmonary delivery of active agents include the convenience of patient self-administration, the potential for reduced drug side-effects, ease of delivery by inhalation, the elimination of needles, and the like. Many clinical studies with inhaled proteins, peptides, DNA and small molecules have demonstrated that efficacy can be achieved both within the lungs and systemically. However, many molecules which require high payload for delivery, and in particular biological molecules, present problems for the development of inhalable formulations. Formulations must provide stability to the biological payload and have scalable manufacturability while also maintaining desirable physical characteristics to facilitate delivery into the lungs of a patient.

### BRIEF SUMMARY OF THE DISCLOSURE

In view of the foregoing, provided herein is a dry powder formulation including a plurality of microparticles, the microparticles comprising leucine; about 0.5% to about 10% trileucine by weight; and an active agent, wherein the leucine and the trileucine are present at a ratio of leucine:trileucine of about 0.1:1 to about 30:1 by weight. In certain embodiments, the dry powder formulation has a compressed bulk density of about 0.4-1.0 g/cm³.

Also provided herein is a method of preparing a dry powder formulation, comprising: preparing a liquid feedstock comprising: leucine; about 0.1 mg/mL to about 6 mg/mL trileucine; an active agent; and a liquid solvent; wherein the leucine and the trileucine are present at a concentration ratio of leucine:trileucine of about 0.1:1 to about 30:1; atomizing the liquid feedstock; and drying the atomized liquid feedstock to form a plurality of microparticles.

In further embodiments, provided herein is a method for preparing a dry powder formulation comprising a plurality of microparticles having a compressed bulk density of about 0.4 to about 1.0 g/cm3, the method comprising: incorporating leucine and trileucine into the dry powder formulation at a ratio of leucine:trileucine of about 0.1:1 to about 30:1 by weight.

Also provided herein is a method for preparing a dry powder formulation comprising a plurality of microparticles having a specific surface area of about 5 to about 10 m²/g, the method comprising: incorporating leucine and trileucine into the dry powder formulation at a ratio of leucine:trileucine of about 0.1:1 to about 30:1 by weight.

In further embodiments, provided herein is a method for preparing a dry powder formulation comprising a plurality of microparticles, wherein the mass median aerodynamic diameter (MMAD) of the microparticles is about 2 μm to about 4 μm when provided in an aerosol form, the method comprising: incorporating leucine and trileucine into the dry powder formulation at a ratio of leucine:trileucine of about 0.1:1 to about 30:1 by weight.

In still further embodiments, provided herein is a method for delivery of a dry powder formulation to the lungs of a mammalian patient, the method comprising administering to the mammalian patient by inhalation the dry powder formulations provided herein, in an aerosol form.

Also provided herein is a method for treating a medical condition in a mammalian patient, comprising administering to the mammalian patient by inhalation the dry powder formulations as described herein, in an aerosol form.

In additional embodiments, the dry powder formulations described herein can be used in a method of treatment, wherein the formulation is to be administered by inhalation.

### BRIEF DESCRIPTION OF DRAWINGS

The foregoing and other features and aspects of the present technology can be better understood from the following description of embodiments and as illustrated in the accompanying drawings. The accompanying drawings, which are incorporated herein and form a part of the specification, further serve to illustrate the principles of the present technology. The drawings are not necessarily to scale.

FIG. 1 shows microparticles from a dry powder formulation in accordance with embodiments hereof.

FIG. 2A shows the results of compressed bulk density as a function of leucine and trileucine in the dry powder formulations.

FIG. 2B shows the filling of capsules with dry powder formulations described herein.

FIG. 3 shows the results of specific surface area measured using BET, in m²/g, for microparticles of dry powder formulations in accordance with embodiments hereof.

FIG. 4 shows the indirect correlation of moisture content with leucine concentration.

FIGS. 5A-5D show surface rugosity of microparticles as detected by SEM.

FIG. 6 shows the correlation between fine particle fraction (FPF) and leucine and trileucine wt % values.

FIG. 7 shows the correlation between device deposition and leucine and trileucine wt % values.

FIG. 8 shows the correlation between fine particle fraction (FPF) and leucine and trileucine wt % values.

FIGS. 9A-9B show the saturation rate modelled at different leucine and trileucine concentration combinations.

FIG. 10A shows the number of sub-visible particles following reconstitution of a formulation comprising 40% (w/w) Fab₁ and varying concentrations of polysorbate-80 (PS-80) to a solution concentration of Fab₁ of 30 mg/ml (in the Figure “≥” comprises an upper size limit of 200 μm)

FIG. 10B shows the number of sub-visible particles following reconstitution of a formulation comprising 40% (w/w) Fab₁ and varying concentrations of PS-80 to a solution concentration of Fab₁ of 2.5 mg/ml (in the Figure “≥” comprises an upper size limit of 200 μm)

FIG. 11A shows the number of sub-visible particles following reconstitution of a formulation comprising 40% (w/w) Fab₁ and varying concentrations of poloxamer-188 to a solution concentration of Fab₁ of 30 mg/ml (in the Figure “≥” comprises an upper size limit of 200 μm)

FIG. 11B shows the number of sub-visible particles following reconstitution of a formulation comprising 40% (w/w) Fab₁ and varying concentrations of poloxamer-188 to a solution concentration of Fab₁ of 2.5 mg/ml (in the Figure “≥” comprises an upper size limit of 200 μm)

FIG. 12A shows the moisture content % of a formulation comprising 40% (w/w) Fab₁ and 1.1% PS-80 following storage for 1 or 3 months at 40° C. and 75% relative humidity (40/75) and for 3 months at 25° C. and 60% relative humidity (25/60)

FIG. 12B shows the particle size distribution (PSD) of a formulation comprising 40% (w/w) Fab₁ and 1.1% PS-80 following storage for 1 or 3 months at 40° C. and 75% relative humidity (40/75) and for 3 months at 25° C. and 60% relative humidity (25/60)

FIG. 12C shows the particle morphology of a formulation comprising 40% (w/w) Fab₁ and 1.1% PS-80 following storage for 1 or 3 months at 40° C. and 75% relative humidity (40/75) and for 3 months at 25° C. and 60% relative humidity (25/60)

FIG. 13A shows the moisture content % of a formulation comprising 1% (w/w) Fab₁ and 1.1% PS-80 following storage for 1 or 3 months at 40° C. and 75% relative humidity (40/75) and for 3 months at 25° C. and 60% relative humidity (25/60)

FIG. 13B shows the particle size distribution (PSD) of a formulation comprising 1% (w/w) Fab₁ and 1.1% PS-80 following storage for 1 or 3 months at 40° C. and 75% relative humidity (40/75) and for 3 months at 25° C. and 60% relative humidity (25/60)

FIG. 13C shows the particle morphology of a formulation comprising 1% (w/w) Fab₁ and 1.1% PS-80 following storage for 1 or 3 months at 40° C. and 75% relative humidity (40/75) and for 3 months at 25° C. and 60% relative humidity (25/60)

FIG. 14A shows the number of sub-visible particles following reconstitution of a formulation comprising 40% Fab₁ and 1.1% PS-80 (w/w) to a solution concentration of Fab₁ of 30 mg/ml, following storage at 40/75 for 1 or 3 months and 25/60 for 3 months

FIG. 14B shows the number of sub-visible particles following reconstitution of a formulation comprising 1% Fab₁ and 1.1% PS-80 (w/w) to a solution concentration of Fab₁ of 0.75 mg/ml, following storage at 40/75 for 1 or 3 months and 25/60 for 3 months

### DETAILED DESCRIPTION

It should be appreciated that the particular implementations shown and described herein are examples and are not intended to otherwise limit the scope of the application in any way.

The published patents, patent applications, websites, company names, and scientific literature referred to herein are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

Technical and scientific terms used herein have the meaning commonly understood by one of skill in the art to which the present application pertains, unless otherwise defined. Reference is made herein to various methodologies and materials known to those of skill in the art.

As described herein, dry powder formulations are provided for the stabilization and delivery of pharmaceutical active agents. Suitably, the dry powder formulations are formulated for pulmonary delivery, including via inhalation via a dry powder inhaler (DPI).

As used herein a “dry powder formulation” refers to a formulation that includes a plurality of solid microparticles in a powder composition that suitably contains less than about 20% moisture, more suitably less than 10% moisture, less than about 5-6% moisture, or less than about 3% moisture. As described herein, dry powder formulations can be utilized for delivery via inhalation to a patient. In other embodiments, the dry powder formulations can be reconstituted and administered in a liquid form, either orally, intravenously, parenterally, etc. As described herein, an advantage of the dry powder formulations provided is the increased throughput for improved manufacturability. A further advantage is that the formulation platform described herein provides for a high compressed bulk density. This means that a greater mass of powder can be packaged per delivery unit (e.g. within a capsule). This means that a high dose of active agent can be delivered per unit delivery to the subject. This surprising advantage may improve patient compliance by lowering the number of unit doses required to be taken. In addition, the high compressed bulk density may enable higher dose of active agent to be delivered, increasing the top-end of administered dose range. This may enable the delivery of active agents at therapeutically effective doses where this was not previously possible.

A “microparticle” as used herein refers to a solid particle having a size mass mean diameter (MMD) of less than 20 μm. Mass mean diameter is a measure of the mean particle size of the microparticles, measured using a suitable method, including for example centrifugal sedimentation, electron microscopy, light scattering, laser diffraction, etc.

The dry powder formulations described herein suitably contain a plurality of microparticles. As used herein “plurality” refers to 2 or more of an item, and suitably refers to 5 or more, 10 or more, 50 or more, 100 or more, 500 or more, 1000 or more, etc.

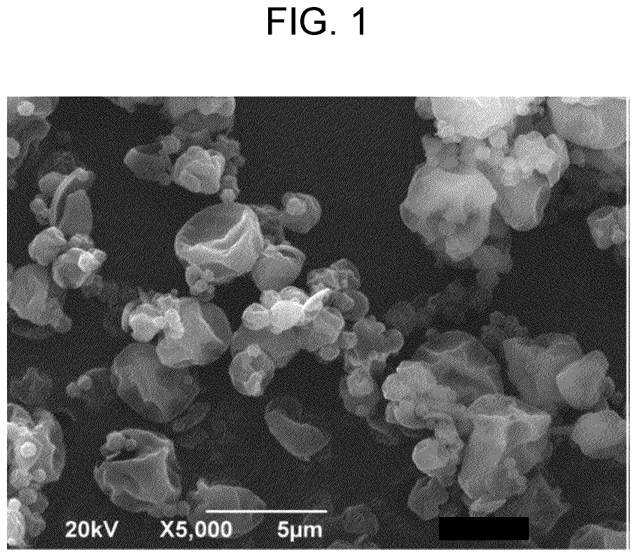

In embodiments, the dry powder formulations include a plurality of microparticles, the microparticles suitably comprise leucine; about 0.5% to about 10% trileucine by weight; and an active agent. FIG. 1 shows a scanning electron micrograph of microparticles of an exemplary dry powder formulation provided herein. In further embodiments, the dry powder formulations including a plurality or microparticles suitably comprise about 1% to about 25% leucine; about 1% to about 10% trileucine; and an active agent.

As used herein “leucine,” whether present as a single amino acid or as an amino acid component of a peptide, refers to the amino acid leucine (C₆Hi₃NO₂), which may be a racemic mixture or in either its D- or L-form, as well as modified forms of leucine (i.e., where one or more atoms of leucine have been substituted with another atom or functional group). The chemical structure of leucine is provided below:

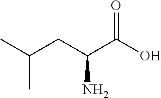

“Trileucine” as utilized herein refers to the chemical compound in which three leucine molecules are linked together in a peptide, as leucine-leucine-leucine (Leu-Leu-Leu), C₁₈H₃₅N₃O₄. The chemical structure of trileucine is provided below:

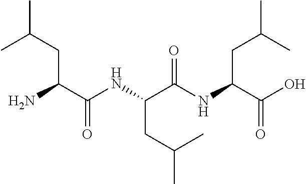 [SMILES: CC(C)C[C@H](NC(=O)[C@H](CC(C)C)NC(=O)[C@@H](N)CC(C)C)C(=O)O]

The amounts of leucine and trileucine provided herein, unless otherwise stated, are provided as weight percentages (wt %) of the formulations. As the dry powder formulations contain substantially little if any water, the weight components of the dry powder formulations are thus dry weight percentages of the final formulations.

In embodiments of the formulation comprising leucine; trileucine; and an active agent, the leucine and trileucine are kept at a desired ratio range that provides the improved compressed bulk density characteristics described herein, as well as providing the desired microparticle characteristics that allow for improved storage and delivery. In embodiments, the weight ratio of leucine and trileucine in the microparticles, i.e., leucine:trileucine, is about 0.05:1 to about 40:1, and more suitably the leucine and the trileucine are present at a weight ratio of leucine:trileucine of about 0.1:1 to about 30:1. In further embodiments, the leucine and the trileucine are present at a weight ratio of leucine:trileucine of about 0.1:1 to about 25:1, about 0.5:1 to about 20:1, about 1:1 to about 20:1, about 1:1 to about 15:1, about 1:1 to about 12:1, about 1:1 to about 10:1, about 1:1 to about 7:1, about 1:1 to about 6:1, or about 1:1:, about 2:1, about 3:1, about 4:1, about 5:1, about 5.1:1:, about 5.2:1 about 5.25:1, about 5.3:1, about 5.4:1, about 5.5:1, about 5.75:1 or about 6:1.

Unless otherwise stated, the ratios described herein are expressed as ratios by weight % (w/w-also referred to as a “weight ratio”), that is, weight of leucine:weight of trileucine in the formulations described herein. The ratios are achieved by providing a desired mg/mL concentration of leucine and trileucine in a feedstock, and then drying to remove the feedstock solvent resulting in an atomized microparticle where the starting concentration ratio (expressed in mg/mL), is maintained as a final ratio of leucine:trileucine by weight.

Exemplary weight percentages for leucine and trileucine that can be utilized in the dry powder formulations to achieve these ratios are described herein. Suitably, the dry powder formulations comprise about 5% to about 15% leucine and about 1% to about 5% trileucine. In embodiments, the dry powder formulations comprise about 8% to about 11% leucine and about 2% to about 4% trileucine, and in embodiments, the dry powder formulations comprise about 10.5% leucine and about 2% trileucine.

In exemplary embodiments, the dry powder formulations comprise about 0.5% to about 10% trileucine by weight, more suitably about 1% to about 10%, 1% to about 9%, about 1% to about 8%, about 1% to about 7%, about 1% to about 6%, about 1% to about 5%, about 2% to about 10%, about 2% to about 9%, about 2% to about 8%, about 2% to about 7%, about 2% to about 6%, about 2% to about 5%, about 2% to about 4%, or about 1%, about 1.5%, about 2%, about 2.5%, about 3%, about 3.5%, about 4%, about 4.5%, about 5%, about 5.5%, or about 6%, trileucine, by weight.

In exemplary embodiments, the dry powder formulations comprise about 1% to about 25% leucine by weight, more suitably about 2% to about 20%, about 3% to about 20%, about 4% to about 20%, about 5% to about 20%, about 5% to about 15%, about 7% to about 12%, about 8% to about 11%, about 9% to about 11%, about 10% to about 11%, or about 5%, about 6%, about 7%, about 8%, about 8.5%, about 9%, about 10%, about 10.5%, about 11%, about 11.5%, about 12%, about 12.5% or about 13%, leucine by weight.

In suitable embodiments, the dry powder formulations comprise about 8% to about 11% leucine and about 2% to about 4% trileucine by weight, more suitably about 9% to about 11% leucine, and about 2% to about 3% trileucine by weight. In exemplary embodiments, the dry powder formulations comprise about 10.5% leucine and about 2% trileucine by weight.

As described herein, it has been surprisingly found that the use of the combination of leucine and trileucine in a dry powder formulation allows for the reduction in the overall amount of leucine and trileucine required to prepare microparticles, as compared to dry powder formulations that contain only one of these components, while still providing the desired stability. In certain embodiments, the formulations described herein have increased compressed bulk density in comparison to formulations in the art, which may enable the delivery of a higher concentration of an active agent to the lungs of a patient following inhalation. These improved characteristics appear to be related to the incorporation of leucine and trileucine into the microparticles.

An exemplary process of preparing a dry powder formulation, in accordance with embodiments hereof, may take place as follows. A liquid feedstock containing the desired final components of the dry powder formulation are atomized using an atomizer, to a fine mist. The mist is then dried as described herein. The atomized droplets contain the dissolved components, initially as a liquid droplet. As the droplet dries, different components of the formulation begin to saturate and precipitate at varying rates. As described herein, a shell begins to form around an outer surface of the microparticles of the dry powder formulations. This shell suitably includes the leucine and trileucine components at an outer surface of the shell. It should be noted that leucine and trileucine become preferentially located at an outer surface of the microparticles, while smaller amounts of leucine and trileucine can also found throughout the microparticles. In embodiments, a higher concentration of leucine and trileucine are suitably found at or near the surface of the microparticles, rather than near the center of the microparticles. In embodiments, the center of the microparticles contain a substantial amount of the active agent, along with other excipient components as described herein, suitably in an amorphous form. As used herein, a “substantial amount” of the active agent means at least about 60% of the active agent (i.e., of the total active agent in the formulation) is located at or near the center of the microparticles, suitably at least about 70%, and more suitably at least about 75%, at least about 80%, at least about 85%, at least about 90%, at least about 95%, and in embodiments about 95%-100%, of the active agent is located at or near the center of the microparticles.

In further embodiments, the microparticles contain leucine and trileucine located substantially throughout the microparticles, but with higher amounts at or near the surface of the microparticles. As used herein “substantially throughout the microparticles” means that the leucine and/or trileucine are located in a gradient from the outer surface of the microparticles toward the center of the microparticles, but suitably with decreasing amounts of the leucine and/or trileucine as you move toward the center, and in embodiments, no leucine or trileucine are found at the center of the microparticles where the active agent is located. In other embodiments, the amounts and leucine and trileucine can be substantially uniform throughout a cross-section of the microparticles.

In embodiments, substantially each of the microparticles of the dry powder formulations comprise leucine and trileucine. That is, suitably at least about 60% of the microparticles contain leucine and trileucine, or at least about 70%, and more suitably at least about 75%, at least about 80%, at least about 85%, at least about 90%, at least about 95%, and in embodiments about 95%-100%, of the microparticles comprise leucine and trileucine. In embodiments each of the microparticles of the dry powder formulations comprise leucine and trileucine.

In additional embodiments, leucine and/or trileucine can be found in the dry powder formulations, but not contained within or associated with a microparticle of the formulation. Thus, in embodiments, free leucine and/or trileucine that is not associated with a microparticle can be found in the dry powder formulations. However, in general, the amount of free leucine and/or trileucine (i.e., not associated with a microparticle) is on the order of less than about 10%, less than about 5%, less than about 1%, and more suitably less than about 0.1% of the total amount of leucine and/or trileucine in the formulations.

In exemplary embodiments, the dry powder formulations described herein have a compressed bulk density that allows for the delivery of a large amount of active agent. “Compressed bulk density” refers to the mass per unit volume (suitably g/cm³) of a powder when measured under the following conditions. A suitable assay for measuring compressed bulk density (cBD, or CBD) is described in the examples (see, e.g., Example 1). Suitably, the compressed bulk density (CBD) of the powders is measured using a density analyzer, such as a GeoPyc® Model 1360 density analyzer (Micromeritics, Norcross, Ga.). Powder samples are suitably prepared in a low humidity environment (<5% RH), before transfer into the density analyzer sample chamber that has been purged with nitrogen gas. The net weight of the powder sample is recorded, and then a compression force of 10-14N, suitably 12N, is applied to the sample by a plunger, at a rate of 250-350 consolidation steps per second, suitably 300 consolidation steps per second. The linear distance travelled by the plunger for each consolidation step is translated into a volume displacement of the powder sample. An average of the measurements from each consolidation step is then transformed into a calculated bulk density value for the dry powder formulation, expressed in g/cm³.

In certain embodiments, the compressed bulk density of a dry powder formulation described herein is at least 0.4 g/cm³, and suitably between about 0.4 g/cm³ to about 1.0 g/cm³, and more suitably about 0.4-0.9 gm/cm³, about 0.4-0.8 gm/cm³, about 0.5-0.8 gm/cm³, about 0.6-0.8 gm/cm³, or about 0.4 gm/cm³, about 0.5 gm/cm³, about 0.6 gm/cm³, about 0.7 gm/cm³, or about 0.8 gm/cm³. In certain embodiments, the compressed bulk density of a dry powder formulation described herein is from about 0.4 gm/cm³ to about 0.9 gm/cm³. In certain embodiments, the compressed bulk density of a dry powder formulation described herein is from about 0.5 gm/cm³ to about 0.8 gm/cm³.

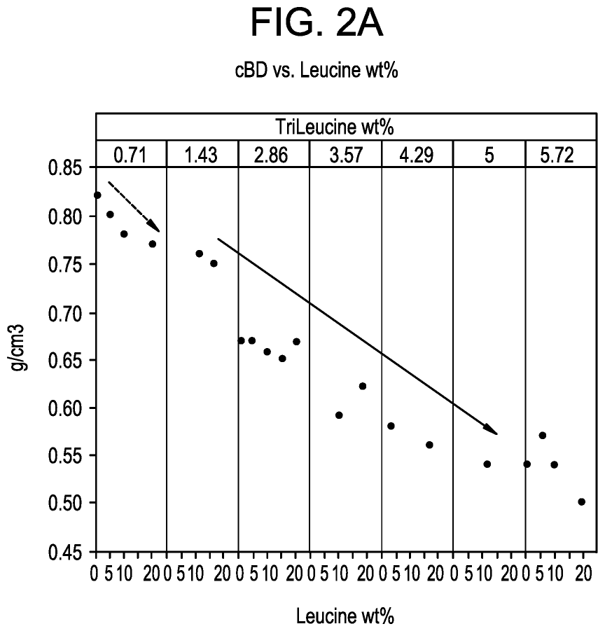

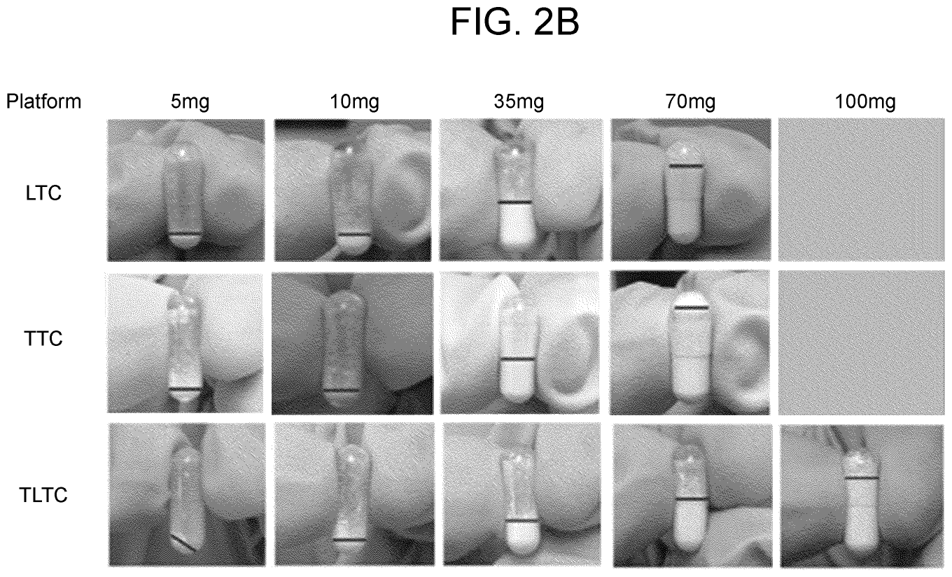

FIG. 2A shows the results of compressed bulk density as a function of leucine and trileucine in the dry powder formulations described herein. Each of the columns represents an amount of trileucine in the formulations. Within each column, the amount of leucine is increased from about 1% to about 20%. As shown, increasing the amount of trileucine results in a lower compressed bulk density, and increasing leucine within each group also reduces the compressed bulk density. To achieve a compressed bulk density of between about 0.5 g/cm³ to about 0.8 g/cm³ the amount of trileucine should be maintained at below 4% by weight.

Various active agents can be formulated in the dry powder formulations described herein. As used herein, an “active agent” refers to a pharmacologically active organic or inorganic compound that acts on a desired target with a mammalian patient, including a human, to treat, improve, ameliorate, or cure, a symptom, disease, infection, condition, etc., of the patient.

Amounts of active agents to be included in the dry powder formulations suitably are in the range of about 10%-80%, by weight, more suitably about 20%-70%, about 30%-50%, or about 30%, about 35%, about 40%, about 45%, about 50%, about 55%, about 60%, etc.

Exemplary active agents include small molecules. As used herein, the term “small molecule” refers to a chemically synthesized, low molecular-weight pharmaceutical, therapeutic and/or diagnostic agent (examples of the latter being markers, dyes, etc.), that generally has a molecular weight of less than about 10 kD, suitably less than about 5000 Daltons, and more suitably less than about 1000 Daltons, for example about 100 to about 900 Daltons, about 200 to about 800 Daltons, about 300 to about 700 Daltons, about 400 to about 600 Daltons, or about 500 Daltons, as well as salts, esters, and other pharmaceutically acceptable forms of such compounds.

In additional embodiments, the active agent can be a biologic. As used herein a “biologic” refers to an isolated or synthetically produced natural product, including nucleic acids, amino acids, peptides, polypeptides, and proteins, and suitably includes antibodies, antigen binding fragments, and the like.

The term “polypeptide” refers to a molecule comprising a polymer of amino acids linked together by a peptide bond(s). Polypeptides include polypeptides of any length, including proteins (e.g. having more than 50 amino acids) and peptides (e.g. 2-49 amino acids). Polypeptides include proteins and/or peptides of any activity, function or size, and include secreted proteins, a membrane-anchored protein or an intracellular protein.

Exemplary polypeptides and recombinant polypeptides include enzymes (e.g. proteases, kinases, phosphatases), receptors, transporters, bactericidal and/or endotoxin-binding proteins, structural polypeptides, membrane-bound polypeptides, glycoproteins, globular proteins, immune polypeptides, toxins, antibiotics, hormones, growth factors, blood factors, vaccines or the like. The polypeptides can be peptide hormones, interleukins, tissue plasminogen activators, cytokines, immunoglobulins, including antibodies or functional antigen binding fragments or variants thereof and Fc-fusion proteins. The polypeptide may also be a subunit or domain of a polypeptide, such as a heavy chain or a light chain of an antibody, or a functional fragment or derivative thereof.

In embodiments, the polypeptide is an immunoglobulin molecule, suitably an antibody, or a subunit or domain thereof such as the heavy or light chain of an antibody. The term “antibody” as used herein refers to a protein comprising at least two heavy chains and two light chains connected by disulfide bonds. The term “antibody” includes naturally occurring antibodies as well as all recombinant forms of antibodies, e.g., humanized antibodies, fully human antibodies and chimeric antibodies. Each heavy chain is usually comprised of a heavy chain variable region (VH) and a heavy chain constant region (CH). Each light chain is usually comprised of a light chain variable region (VL) and a light chain constant region (CL). The term “antibody”, however, also includes other types of antibodies such as single domain antibodies, heavy chain antibodies, i.e. antibodies only composed of one or more, in particular two heavy chains, and nanobodies, i.e. antibodies only composed of a single monomeric variable domain. Examples of fragments or derivatives of an antibody include (i) Fab fragments, monovalent fragments consisting of the variable region and the first constant domain of each the heavy and the light chain; (ii) F(ab)2 fragments, bivalent fragments comprising two Fab fragments linked by a disulfide bridge at the hinge region; (iii) Fd fragments consisting of the variable region and the first constant domain CH1 of the heavy chain; (iv) Fv fragments consisting of the heavy chain and light chain variable region of a single arm of an antibody; (v) scFv fragments, Fv fragments consisting of a single polypeptide chain; (vi) (Fv)2 fragments consisting of two Fv fragments covalently linked together; (vii) a heavy chain variable domain; and (viii) multibodies consisting of a heavy chain variable region and a light chain variable region covalently linked together in such a manner that association of the heavy chain and light chain variable regions can only occur intermolecular but not intramolecular.

Examples of active agents include small molecules or biologics that are useful for treating patients that are suffering from or pre-disposed to any disease state, including, but not limited to, cancers (e.g., a breast cancer, a uterine cancer, an ovarian cancer, a prostate cancer, a testicular cancer, a lung cancer, a leukemia, a lymphoma, a colon cancer, a gastrointestinal cancer, a pancreatic cancer, a bladder cancer, a kidney cancer, a bone cancer, a neurological cancer, a head and neck cancer, a skin cancer, a sarcoma, an adenoma, a carcinoma and a myeloma); infectious diseases (e.g., bacterial diseases, fungal diseases, parasitic diseases and viral diseases (such as a viral hepatitis, a disease caused by a cardiotropic virus; HIV/AIDS, flu, SARS, and the like)); genetic disorders (e.g., anemia, neutropenia, thrombocytopenia, hemophilia, dwarfism and severe combined immunodeficiency disease (“SCID”); inflammatory and autoimmune disorders (e.g., psoriasis, systemic lupus erythematosus and rheumatoid arthritis, asthma, including severe asthma, moderate asthma or mild asthma, chronic obstructive pulmonary disease, atopic dermatitis and idiopathic pulmonary fibrosis) and neurodegenerative disorders (e.g., various forms and stages of multiple sclerosis, Creutzfeldt-Jakob Disease, Alzheimer's Disease, and the like). In certain embodiments, the active agent is for use in the treatment of severe asthma, such as eosinophilic or non-eosinophilic asthma, optionally low eosinophilic asthma.

Exemplary active agents that can be included in the dry powder formulations described herein include, but are not limited to, inhaled corticosteroids (ICS), long-acting beta agonists (LABA), leukotriene receptor antagonists (LTRA), long-acting anti-muscarinics (LAMA), cromones, short-acting beta agonist (SABA), cytokines including interleukins, hormones, interferons, tissue growth factors, endothelial growth factors, phosphodiesterase (PDE) compounds, VLA-4 inhibitors, bisphosponates, macrolides, antibiotics, fluoroquinolones, aminoglycosides, polymixins, antifungal agents, carbapenems, etc., and where applicable, analogues, agonists, antagonists, inhibitors, and pharmaceutically acceptable salt forms of the above.

In reference to peptides and proteins, it is intended to encompass synthetic, native, glycosylated, unglycosylated, pegylated forms, and biologically active fragments and analogs thereof. Active agents further include nucleic acids, as bare nucleic acid molecules, vectors, associated viral particles, plasmid DNA or RNA or other nucleic acid constructions of a type suitable for transfection or transformation of cells, i.e., suitable for gene therapy including antisense, siRNA, miRNA, etc. Further, an active agent may comprise live, attenuated or killed viruses suitable for use as vaccines.

In embodiments, the active agent is an anti-TSLP antibody or antibody variant, and suitably an anti-TSLP antigen binding fragment thereof. Thymic stromal lymphopoietin (TSLP) is an epithelial-cell—derived cytokine that plays a role in initiating allergic inflammation. The anti-TSLP antigen binding fragment described herein (referred to as Fab 1) may be useful in the treatment of asthma. Exemplary Fab 1 sequences include:

HCDR1 FAB1
(SEQ ID NO: 1)
Thr Tyr Gly Met His
HCDR2 FAB1
(SEQ ID NO: 2)
Val Ile Trp Tyr Asp Gly Ser Asn Lys His
Tyr Ala Asp Ser Val Lys Gly
HCDR3 FAB1
(SEQ ID NO: 3)
Ala Pro Gln Trp Glu Leu Val His Glu Ala
Phe Asp Ile
HEAVY CHAIN VH FAB1
(SEQ ID NO: 4)
Gln Met Gln Leu Val Glu Ser Gly Gly Gly Val
Val Gln Pro Gly Arg Ser Leu Arg Leu Ser Cys
Ala Ala Ser Gly Phe Thr Phe Arg Thr Tyr Gly
Met His Trp Val Arg Gln Ala Pro Gly Lys Gly
Leu Glu Trp Val Ala Val Ile Trp Tyr Asp Gly
Ser Asn Lys His Tyr Ala Asp Ser Val Lys Gly
Arg Phe Thr Ile Thr Arg Asp Asn Ser Lys Asn
Thr Leu Asn Leu Gln Met Asn Ser Leu Arg
Ala Glu Asp Thr Ala Val Tyr Tyr Cys Ala Arg
Ala Pro Gln Trp Glu Leu Val His Glu Ala Phe
Asp Ile Trp Gly Gln Gly Thr Met Val Thr Val
Ser Ser
LCDR1 FAB1
(SEQ ID NO: 5)
Gly Gly Asn Asn Leu Gly Ser Lys Ser Val His
LCDR2 FAB1
(SEQ ID NO: 6)
Asp Asp Ser Asp Arg Pro Ser
LCDR3 FAB1
(SEQ ID NO: 7)
Gln Val Trp Asp Ser Ser Ser Asp His Val Val
LIGHT CHAIN VL FAB1
(SEQ ID NO: 8)
Ser Tyr Val Leu Thr Gln Pro Pro Ser Val Ser
Val Ala Pro Gly Gln Thr Ala Arg Ile Thr Cys
Gly Gly Asn Asn Leu Gly Ser Lys Ser Val His
Trp Tyr Gln Gln Lys Pro Gly Gln Ala Pro Val
Leu Val Val Tyr Asp Asp Ser Asp Arg Pro Ser
Trp Ile Pro Glu Arg Phe Ser Gly Ser Asn Ser
Gly Asn Thr Ala Thr Leu Thr Ile Ser Arg Gly
Glu Ala Gly Asp Glu Ala Asp Tyr Tyr Cys Gln
Val Trp Asp Ser Ser Ser Asp His Val Val Phe
Gly Gly Gly Thr Lys Leu Thr Val Leu
FAB1 VARIABLE HEAVY CHAIN
(SEQ ID NO: 9)
cagatgcagt tggttgaatc tggtggcggc gtggtgcagc ctggcagatc tctgagactg 60
tcttgtgccg cctccggctt caccttcaga acctacggaa tgcactgggt ccgacaggcc 120
cctggcaaag gattggaatg ggtcgccgtg atttggtacg acggctccaa caagcactac 180
gccgactccg tgaagggcag attcaccatc accagagaca actccaagaa caccctgaac 240
ctgcagatga actccctgag agccgaggac accgccgtgt actattgtgc tagagcccct 300
cagtgggaac tcgtgcatga ggcctttgac atctggggcc agggaacaat ggtcaccgtc 360
tcctca 366
FAB1 VARIABLE LIGHT CHAIN
(SEQ ID NO: 10)
tcatatgttc ttacacaacc accgtcggtt tcggttgctc caggacaaac agctcgaatt 60
acatgcggag gaaacaacct cggatcgaag tcggttcact ggtatcaaca aaagccagga 120
caagctccag ttctcgtggt gtacgatgat tcagatcgac catcatggat cccagagcga 180
ttctcaggat caaactcggg aaatactgcc acgctcacaa tttcacgcgg agaagcggga 240
gatgaagctg attactattg ccaagtgtgg gactcgtcgt cagatcatgt tgttttcgga 300
ggtggaacaa agctcacagt gctc 324

In suitable embodiments, the dry powder formulations described herein further comprise a glass stabilization agent to aid in stabilizing the formulation, and in particular, in stabilizing the active agent. A “glass stabilization agent” refers to an excipient that stabilizes an active agent (suitably a polypeptide) in a dry powder formulation, suitably by substituting for water at the active agent surface during drying, or otherwise impeding the degradation process, and forms an amorphous solid that includes the active agent. Examples of glass stabilization agents include amorphous saccharides, polymeric sugars, buffers, salts, or synthetic polymers (e.g., poly-L-glycolic acid), as well as mixtures of such components. In suitable embodiments, the glass stabilization agent is an amorphous saccharide. In additional embodiments, the glass stabilization agent is a buffer. In still further embodiments, the formulations described herein can include both an amorphous saccharide and a buffer, which together or separately may act as a glass stabilization agent.

Exemplary amorphous saccharides for use in the formulations described herein include, but are not limited to, trehalose, sucrose, raffinose, inulin, dextran, mannitol, and cyclodextrin. Suitably the amorphous saccharide is present at about 30% to about 70% (weight percentage) of the dry powder formulation. In further embodiments, the amorphous saccharide is present at about 30% to about 65%, about 35% to about 65%, about 35% to about 60%, about 40% to about 60%, about 30% to about 50%, or about 30%, about 35%, about 40%, about 45%, about 50%, about 55%, or about 60%. Suitably the amorphous saccharide is trehalose, and is present in the formulations at about 30%-60%, more suitably about 35%-55%, or about 35%, about 40%, about 45% or about 50%, of the weight of the dry powder formulation.

Exemplary buffers that can be included in the dry powder formulations, suitably as glass stabilization agents, include various citrate buffers (such as sodium citrate), a phosphate buffer, a histidine buffer, a glycine buffer, an acetate buffer, and a tartrate buffer, as well as combinations of such buffers. Amounts of the buffers that can be included in the dry powder formulations can range from about 0.1% to about 20%, more suitably about 0.5% to about 15%, about 1% to about 10%, about 2% to about 8%, about 3% to about 7%, or about 1%, about 2%, about 3%, about 4%, about 5%, about 6%, about 7%, about 8%, about 9% or about 10%.

Buffers also provide control of the pH of the dry powder formulations, suitably maintaining a pH of between about pH 5 and about 8, for example, about pH 5 to about pH 6, about pH 5.5 to about pH 6.5, about pH 6 to about pH 7, about pH 6.5 to about pH 7.5, or about pH 7 to about pH 8.

In additional embodiments, dry powder formulations are provided that comprise about 30%-50%, trehalose, about 10%-11% leucine, about 1%-3% trileucine, about 8%-9% citrate buffer and an active agent, more suitably about 39% trehalose, about 10.5% leucine, about 2% trileucine, about 8.5% citrate buffer and an active agent.

In additional embodiments, dry powder formulations are provided that consist essentially of about 30%-50% of an amorphous saccharide, leucine, about 0.5% to about 10% trileucine, about 1% to about 10% of a buffer, and an active agent, wherein the wherein the leucine and the trileucine are present at a concentration ratio of leucine:trileucine of about 0.1:1 to about 30:1. In additional embodiments, dry powder formulations are provided that consist essentially of about 30%-50% of an amorphous saccharide, about 8% to about 11% leucine, about 2% to about 4% trileucine, about 1% to about 10% of a buffer, and an active agent. Additional dry powder formulations are provided that consist essentially of about 35%-45% trehalose, about 9% to about 11% leucine, about 2% to about 3% trileucine, about 2% to about 85 citrate buffer, and an active agent. In further embodiments, the dry powder formulations consist essentially of about 39% trehalose, about 10.5% leucine, about 2% trileucine, about 8.5% citrate buffer, and an active agent.

In compositions and formulations that “consist essentially” of the recited ingredients, such compositions and formulations contain the recited components and those that do not materially affect the basic and novel characteristics of the claimed formulations. Components that do not materially affect the basic and novel characteristics of the claimed formulations are those that do not limit the ability of the leucine and trileucine to stabilize the dry powder formulations. Suitably, compositions and formulations that consist essentially of the recited ingredients specifically exclude other amino acids or tripeptide amino acids, but can include additional sugars, buffers, etc.

In exemplary embodiments, a dry powder formulation is provided that comprises about 30-50%, trehalose, about 10%-11% leucine, about 1%-3% trileucine, about 8%-9% citrate buffer and about 30-50% of anti-TSLP antibody antigen binding fragment, more suitably about 39% trehalose, about 10.5% leucine, about 2% trileucine, about 8.5% citrate buffer and about 40% of anti-TSLP antibody antigen binding fragment. In certain embodiments, the anti-TSLP antibody antigen binding fragment is Fab 1.

In further exemplary embodiments, a dry powder formulation is provided that consists essentially of about 30-50%, trehalose, about 10%-11% leucine, about 1%-3% trileucine, about 8%-9% citrate buffer and about 30-50% of anti-TSLP antibody antigen binding fragment, more suitably about 39% trehalose, about 10.5% leucine, about 2% trileucine, about 8.5% citrate buffer and about 40% of anti-TSLP antibody antigen binding fragment.

The microparticles that make up the dry powder formulations described herein suitably have a specified mass median aerodynamic diameter (MMAD) when provided in aerosol form. The microparticles may also have a specified optical volume mean diameter (oVMD). oVMD may also be referred to as particle size distribution (PSD or pPSD).

As used herein, “mass median aerodynamic diameter” or “MMAD” is a measure of the aerodynamic size of a dispersed microparticle. The aerodynamic diameter is used to describe an aerosolized powder in terms of its settling behavior and is the diameter of a unit density sphere having the same settling velocity, in air, as the microparticle. The aerodynamic diameter encompasses particle shape, density and physical size of a microparticle. As used herein, MMAD refers to the midpoint or median of the aerodynamic particle size distribution of an aerosolized powder determined by cascade impaction, unless otherwise indicated. Suitably the microparticles of the dry powder formulations provided herein have a mass median aerodynamic diameter (MMAD) of at least 1 μm or greater, more suitably about 1 μm to about 10 μm, about 2 μm to about 8 μm, about 2 μm to about 7 μm, about 2 μm to about 6 μm, about 2 μm to about 5 μm, about 2 μm to about 4 μm, about 3 μm to about 7 μm, about 4 μm to about 7 μm, about 3 μm to about 6 μm, or about 2 μm, about 3 μm, about 4 μm, about 5 μm, about 6 μm, or about 7 μm.

Suitably, the fine particle fraction (the fraction of particles emitted from an inhalation device having an aerodynamic particle diameter of less than 5 μm of the dry powder formulations described herein is ≥50%, more suitably ≥60%. This fine particle fraction (FPF) may contribute to a low device retention of the dry powder formulations of less than 20%, suitably less than 15%, less than 10%, or less than 5%, remaining in a device following delivery to a patient.

In additional embodiments, the microparticles suitably have an equivalent optical volume mean diameter (oVMD) of about 0.5 μm to about 7 μm. Equivalent optical volume mean diameter (oVMD) refers the mean diameter of a sphere that best approximates a specific optical interaction of the microparticle with light, where half of the microparticles are best approximated by an equivalent sphere smaller, and half of the microparticles are best approximated by an equivalent sphere larger than the mean, when measured using a suitable optical technique. In exemplary embodiments, the microparticles have an equivalent optical volume median diameter (oVMD) of about 0.5 μm to about 6 μm, or about 1 μm to about 5 μm, or about 1 μm to about 4 μm, or about 2 μm to about 4.5 μm, or about 2.5 μm to about 4 μm, or about 2 μm to about 4 μm, or about 2 μm to about 3 μm, or about 2 μm to about 3.5 μm, or about 1 μm, about 1.5 μm, about 2 μm, about 2.5 μm, about 3 μm, about 3.5 μm, about 4 μm, about 4.5 μm, or about 5 μm.

As described herein, a high compressed bulk density allows for the delivery of a larger amount of active agent, utilizing the same delivery volume. Certain biological agents may require delivery payloads of as much as 50 mg/dose, or higher, to effective treatment. As shown illustratively in FIG. 2B, the combination of leucine and trileucine can result in a dry powder formulation that has a higher bulk density, and therefore for the same amount of fill weight, takes up substantially less volume.

Exemplary platform formulations shown in FIG. 2B are provided below. LTC indicates a formulation with no Trileucine (TLeu), but containing leucine, trehalose and citrate buffer; TTC indicates a formulation with no Leucine (Leu), but containing trileucine, trehalose and citrate buffer; TLTC indicates the inclusion of both leucine and trileucine, as well as trehalose and citrate buffer. Cit refers to citrate buffer. Tre refers to trehalose.

TABLE 1
Exemplary Platform Formulations

| Platform | % Tre | % Leu | % TLeu | % Cit |
|---|---|---|---|---|
| LTC | 46 | 45 | 0 | 9 |
| TTC | 81 | 0 | 11.2 | 7.8 |
| TLTC | 79 | 10.5 | 2 | 8.5 |

Capsules (size 3 capsules) of each formulation are shown at the respective fill weights in FIG. 2B. As illustrated, for the TLTC formulation, the combination of trileucine and leucine allows for the filling of a capsule with 100 mg of dry powder formulation, while still maintaining some remaining space in the capsule. The other formulations could not be filled above about 70-80 mg fill weight. This represents the dramatic improvement provided by the use of leucine and trileucine in combination to prepare a formulation with a high compressed bulk density, allowing for a high fill weight.

As described herein, the use of leucine and trileucine in the dry powder formulations also results in microparticles having the desired sizes (MMAD), as well as desirable specific surface area (SSA) and roughness, resulting in microparticles that can flow appropriately and be delivered to the lungs using various inhalation platforms.

Specific surface area (SSA) of the microparticles is defined as the total surface area of the microparticles per unit of mass (suitably with units of m²/g). Methods of measuring SSA are known in the art, and include for example Brunauer—Emmett—Teller (BET) measurements using specific surface area evaluation of materials by nitrogen adsorption measured as a function of relative pressure. The surface area is determined by calculating the amount of adsorbate gas corresponding to a monomolecular layer on the surface of the microparticles. The technique measures external area and any pore area evaluations to determine the total specific surface area. Instruments for measuring BET are known in the art.

In embodiments, the specific surface area (SSA) of the microparticles of the dry powder formulations is about 3 m²/g to about 8 m²/g. In suitable embodiments, the SSA of the plurality of microparticles is about 3.5 m²/g -7.5 m²/g, or about 4 m²/g-7 m²/g, or about 4.5 m²/g-7 m²/g, or about 5 m²/g-7 m²/g or about 4.5 m²/g -6 m²/g_{,} ²/or about 5 m²/g-6 m²/g, or about 4 m²/g, about 4.5 m²/g, about 5 m²/g, about 5.5 m²/g, about 6 m²/g, about 6.5 m²/g, or about 7 m²/g.

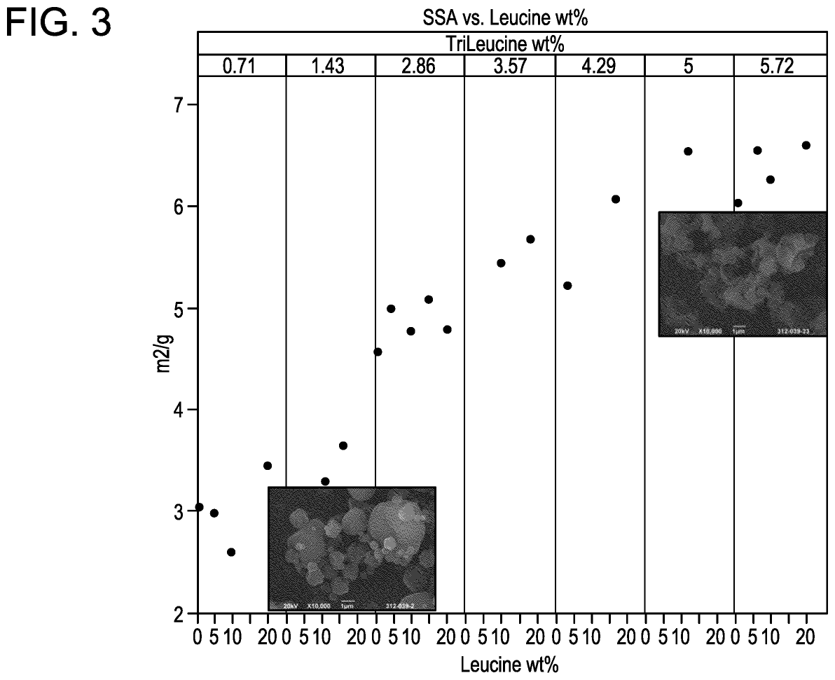

FIG. 3 shows the results of specific surface area measured using BET, in m²/g. Each column within FIG. 3 represents a different amount of trileucine in the formulations. Within each column, the amount of leucine increases from about 1% to about 20%. Inset micrographs demonstrate the physical appearance of the microparticles at low SSA (bottom left) and higher SSA (top right). As shown, at lower wt % trileucine, SSA remains below approximately 5 m²/g, but increases with increasing leucine. Above about 1% trileucine, the SSA increases to greater than 3.0 m²/g, and also increases with increasing percent leucine. SSA values above 5.5 m²/g, and approaching 7.0 m²/g, are achieved with trileucine amounts above about 4%. A desirable range of specific surface area of about 4-7 m²/g can readily be achieved using between about 1-6% trileucine, and amounts of leucine between about 1-20%. As shown, by utilizing an amount of trileucine below about 6%, the amount of leucine can be kept below 10%, even below 5%, and still maintain a desirable SSA and microparticles with a surface roughness. The micrograph at the top left shows the shape of microparticles of the dry powder formulations described herein, exhibiting a desirable size, specific surface are, and surface roughness.

In certain embodiments, the dry powder formulation has a compressed bulk density of about 0.4-1.0 g/cm³. Suitably, the compressed bulk density of the dry powder formulation is about 0.5-0.8 g/cm³. In embodiments, the compressed bulk density of a dry powder formulation described herein is about 0.4-0.9 gm/cm³, about 0.4-0.8 gm/cm³, about 0.5-0.8 gm/cm³, about 0.6-0.8 gm/cm³, or about 0.4 gm/cm³, about 0.5 gm/cm³, about 0.6 gm/cm³, about 0.7 gm/cm³, or about 0.8 gm/cm³. In certain embodiments, the compressed bulk density of a dry powder formulation described herein is from about 0.4 gm/cm³ to about 0.9 gm/cm³. In certain embodiments, the compressed bulk density of a dry powder formulation described herein is from about 0.5 gm/cm³ to about 0.8 gm/cm³.

The dry powder formulations suitably include a glass stabilization agent as described herein, including an amorphous saccharide or a buffer, or the use of both an amorphous saccharide and a buffer. Exemplary amorphous saccharides include those described herein, including trehalose, sucrose, raffinose, inulin, dextran and cyclodextrin. Suitably the amorphous saccharide is present at about 30% to about 70%, and in embodiments is trehalose, suitably present at about 35%-60, or 35%-55%.

Exemplary buffers for use in the dry powder formulations are described herein, and include a citrate buffer, a phosphate buffer and a tartrate buffer. Suitably the buffer is present at about 1% to about 10%, and in embodiments is a citrate buffer. In certain embodiments, the pH of the citrate buffer is from about pH 5.5 to about pH 6.5, such as about pH 5.5, about pH 5.6, about pH 5.7, about pH 5.8, about pH 5.9, about pH 6.0, about pH 6.1, about pH 6.2, about pH 6.3, about pH 6.4 or about pH 6.5. In certain embodiments, the pH of the citrate buffer is about pH 6.4.

In certain embodiments the dry powder formulations described herein comprise a surfactant. As defined herein, “surfactant” refers to a molecule or compound that reduces particle agglomeration, particle adhesion to the surface of a capsule, container walls or valve components of an inhalable delivery device. It has also been found that a surfactant reduces the formation of sub-visible particles (SVPs) upon reconstitution of the formulation. Removing or reducing the formation of SVPs simplifies the analytical characterization of the formulation, as it removes the burden of tracking the formation of SVPs during manufacturing. The analytical characterization of SVPs may involve the development of orthogonal techniques to identify and quantify SVPs for quality control purposes. Thus, removing SVPs or reducing them to acceptable levels removes the necessity of this characterization step from the manufacturing process, streamlining manufacturing. The removal of SVPs may also make dose ranging more predictable, since the kinetics of drug-release from SVPs is unknown. Furthermore, removing SVPs is likely to increase the amount of active agent available to engage pharmacological activity post-reconstitution, which may mean not only that a higher delivered dose can be achieved, but a more accurate prediction of the delivered dose can be calculated. A higher delivered dose may also benefit the patient, for example, by potentially reducing the number or frequency of doses that must be delivered for extracting a pharmacological benefit.

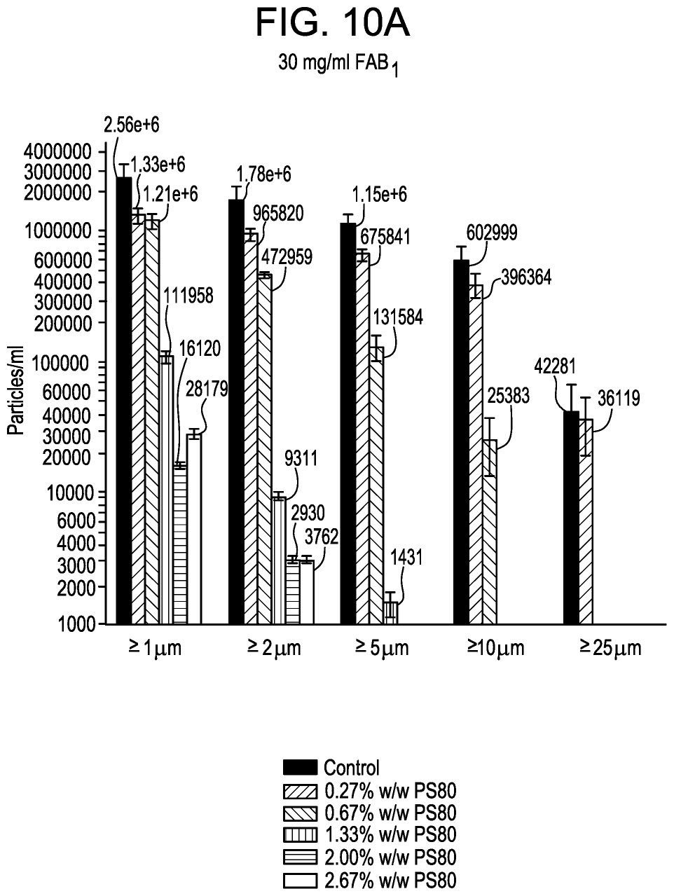

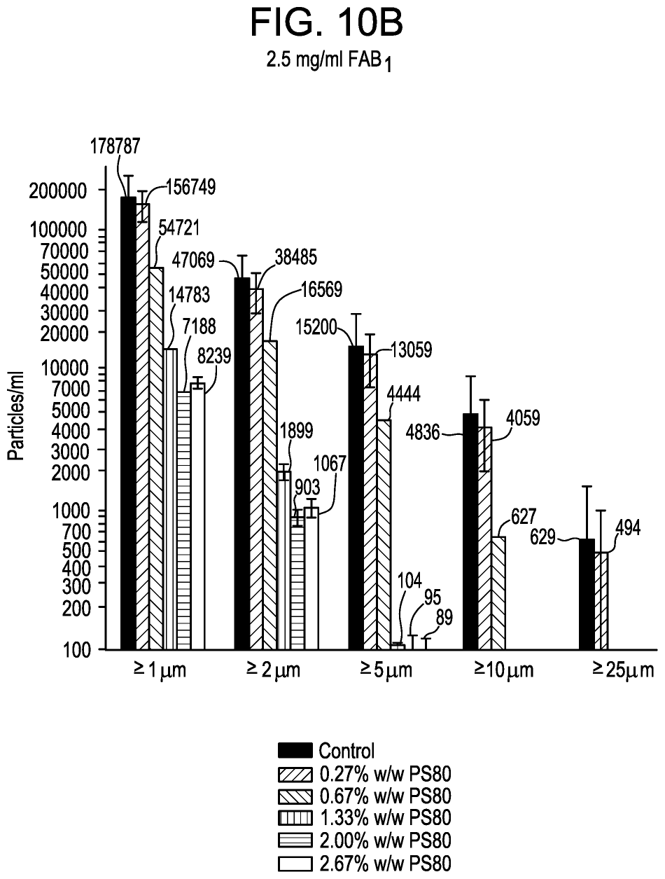

A “sub-visible particle” (“SVP”) is a particle not visible to the naked eye of from about 1 μm to about 200 μm. The presence of sub-visible particles can be inferred on reconstitution of a dry powder formulation by the reconstituted liquid being cloudy. The actual determination of the presence of SVPs can be confirmed using a technique like microflow imaging. Microflow imaging (or MFI), combines microfluidic flow microscopy and high resolution imaging particle analysis to quantify SVP counts. MFI can bin these counts across a particle size range, for example, by binning particles counts in a size range of about 1 to about 200 μm, about 2 μm to about 200 μm, about 5 μm to about 200 μm, about 10 μm to about 200 μm and about 25 μm to about 200 μm). The examples show that the inclusion of a surfactant reduces the presence of SVPs in each particle size range in comparison to a control formulation in which no surfactant is present (e.g. FIG. 10A). Therefore, in certain embodiments, a dry powder formulation disclosed herein comprises a surfactant, wherein upon reconstitution, the number of sub-visible particles in the formulation are decreased. In some embodiments, the number of sub-visible particles are decreased in comparison to an equivalent formulation having no surfactant.

In certain embodiments, the number of SVPs of about 25 μm to about 200 μm in size are decreased to below 30,000 particles per ml, such as 25,000 particles per ml, 20,000 particles per ml, 15,000 particles per ml, 10,000 particles per ml or 5,000 particles per ml. In certain embodiments, the number of SVPs of about 25 μm to about 200 μm in size are decreased to below 1,000 particles per ml. In certain embodiments, the number of SVPs of about 25 μm to about 200 μm in size are decreased to below 1,000 particles per ml. In certain embodiments, the number of SVPs of about 25 μm to about 200 μm in size are decreased to below 100 particles per ml.

In certain embodiments, the number of SVPs of about 10 μm to about 200 μm in size are decreased to below 100,000 particles per ml, such as 90,000 particles per ml, 80,000 particles per ml, 70,000 particles per ml, 60,000 particles per ml, 50,000 particles per ml, 40,000 particles per ml or 30,000 particles per ml. In certain embodiments, the number of SVPs of about 10 μm to about 200 μm in size are decreased to below 10,000 particles per ml. In certain embodiments, the number of SVPs of about 10 μm to about 200 μm in size are decreased to below 1,000 particles per ml. In certain embodiments, the number of SVPs of about 10 μm to about 200 μm in size are decreased to below 100 particles per ml.

In certain embodiments, the number of SVPs of about 5 μm to about 200 μm in size are decreased to below 200,000 particles per ml, such as 180,000 particles per ml, 170,000 particles per ml, 160,000 particles per ml, 150,000 particles per ml or 140,000 particles per ml. In certain embodiments, the number of SVPs of about 5 μm to about 200 μm in size are decreased to below 50,000 particles per ml. In certain embodiments, the number of SVPs of about 5 μm to about 200 μm in size are decreased to below 10,000 particles per ml. In certain embodiments, the number of SVPs of about 5 μm to about 200 μm in size are decreased to below 2,000 particles per ml.

In certain embodiments, the number of SVPs of about 2 μm to about 200 μm in size are decreased to below 1×10⁶ particles per ml, such as 0.8×10⁶ particles per ml, 0.7×10⁶ particles per ml, 0.6×10⁶ particles per ml or 0.5×10⁶ particles per ml. In certain embodiments, the number of SVPs of about 2 μm to about 200 μm in size are decreased to below 100,000 particles per ml. In certain embodiments, the number of SVPs of about 2 μm to about 200 μm in size are decreased to below 50,000 particles per ml. In certain embodiments, the number of SVPs of about 2 μm to about 200 μm in size are decreased to below 10,000 particles per ml.

In certain embodiments, the number of SVPs of about 1 μm to about 200 μm in size are decreased to below 2×10⁶ particles per ml, such as 1.8×10⁶ particles per ml, 1.7×10⁶ particles per ml, 1.6×10⁶ particles per ml or 1.5×10⁶ particles per ml. In certain embodiments, the number of SVPs of about 1 μm to about 200 μm in size are decreased to below 200,000 particles per ml. In certain embodiments, the number of SVPs of about 1 μm to about 200 μm in size are decreased to below 150,000 particles per ml.

In certain embodiments, the number of SVPs of about 25 μm to about 200 μm in size are reduced more than 2-fold, such as more than 3-fold, more than 4-fold, more than 5-fold, more than 6-fold, more than 7-fold, more than 8-fold or more than 9-fold, upon reconstitution, compared to a reference control. In certain embodiments, the number of SVPs of about 25 μm to about 200 μm in size are reduced more than 10-fold upon reconstitution compared to the reference control.

In certain embodiments, the number of SVPs of about 10 μm to about 200 μm in size are reduced more than 2-fold, such as more than 3-fold, more than 4-fold, more than 5-fold, more than 6-fold, more than 7-fold, more than 8-fold or more than 9-fold, upon reconstitution, compared to a reference control. In certain embodiments, the number of SVPs of about 10 μm to about 200 μm in size are reduced more than 10-fold upon reconstitution compared to the reference control.

In certain embodiments, the number of SVPs of about 5 μm to about 200 μm in size are reduced more than 2-fold, such as more than 3-fold, more than 4-fold, more than 5-fold, more than 6-fold, more than 7-fold, more than 8-fold or more than 9-fold, upon reconstitution, compared to a reference control. In certain embodiments, the number of SVPs of about 5 μm to about 200 μm in size are reduced more than 10-fold upon reconstitution compared to the reference control.

In certain embodiments, the number of SVPs of about 2 μm to about 200 μm in size are reduced more than 2-fold, such as more than 3-fold, more than 4-fold, more than 5-fold, more than 6-fold, more than 7-fold, more than 8-fold or more than 9-fold, upon reconstitution, compared to a reference control. In certain embodiments, the number of SVPs of about 2 μm to about 200 μm in size are reduced more than 10-fold upon reconstitution compared to the reference control. In certain embodiments, the number of SVPs of about 2 μm to about 200 μm in size are reduced more than 100-fold upon reconstitution compared to the reference control.

In certain embodiments, the number of SVPs of about 1 μm to about 200 μm in size are reduced more than 2-fold, such as more than 3-fold, more than 4-fold, more than 5-fold, more than 6-fold, more than 7-fold, more than 8-fold or more than 9-fold, upon reconstitution, compared to a reference control. In certain embodiments, the number of SVPs of about 1 μm to about 200 μm in size are reduced more than 10-fold upon reconstitution compared to the reference control.

In certain embodiments the reference control is an equivalent formulation lacking a surfactant. In some embodiments, the formulation is reconstituted in water. In some embodiments, the formulation is reconstituted to an active agent concentration of 30 mg/ml. In some embodiments, the formulation is reconstituted to an active agent concentration of 2.5 mg/ml. In some embodiments, the number of SVPs are determined by microflow imaging (MFI).

Exemplary surfactants suitable for use in the dry powder formulations described herein include, but are not limited to, polysorbate-20 (PS-20), polysorbate-40 (PS-40), polysorbate-60 (PS-60), polysorbate-80 (PS-80) and poloxamer-188. In certain embodiments, the formulations described herein comprise PS-80, suitably at a concentration in the range of from about 0.27% by weight to about 2.7% by weight, suitably from about 0.27% by weight to about 1.33% by weight, suitably from about 0.67% by weight to about 1.33% by weight. In certain embodiments, the formulation comprises PS-80 at a concentration in the range of from about 0.3% by weight to about 3% by weight. In certain embodiments, the formulation comprises PS-80 at a concentration in the range of from about 0.3% by weight to about 2.5% by weight. In certain embodiments, the formulation comprises PS-80 at a concentration in the range of from about 0.5% by weight to about 2.5% by weight. In certain embodiments, the formulation comprises PS-80 at a concentration in the range of from about 0.5% by weight to about 2% by weight. In certain embodiments, the formulation comprises PS-80 at a concentration in the range of from about 0.5% by weight to about 1.5% by weight.

In exemplary embodiments, the formulation comprises PS-80 at a concentration in the range of from about 0.67% to about 1.33%.

In exemplary embodiments, the formulation comprises PS-80 at a concentration of about 0.7% (w/w), about 0.8% (w/w), about 0.9% (w/w), about 1.0% (w/w), about 1.1% (w/w), about 1.2% (w/w), or about 1.3% (w/w). In some embodiments, the formulation comprises PS-80 at a concentration of about 1.1% (w/w).

In exemplary embodiments, the composition comprises PS-80 at a concentration of 0.7%±0.35 (w/w), about 0.8%±0.4 (w/w), about 0.9%±0.45 (w/w), about 1.0%±0.5 (w/w), about 1.1%±0.55 (w/w), about 1.2%±0.6 (w/w), or about 1.3%±0.65 (w/w). In some embodiments, the formulation comprises PS-80 at a concentration of 1.1%±0.55 (w/w).

In exemplary embodiments, the composition comprises PS-80 at a concentration of 0.7%±0.35 (w/w), about 0.8%±0.4 (w/w), about 0.9%±0.45 (w/w), about 1.0%±0.5 (w/w), about 1.1%±0.55 (w/w), about 1.2%±0.6 (w/w), about 1.3%±0.65 (w/w), about 1.4%±0.7 (w/w), about 1.5%±0.75 (w/w), about 1.6%±0.8 (w/w) or about 1.7%±0.75 (w/w).

In certain embodiments, the formulations described herein comprise poloxamer-188, suitably at a concentration in the range of from about 1% by weight to about 10% by weight. In exemplary embodiments, the formulation comprises poloxamer-188 (P188) at a concentration in the range of from about 0.67% to about 2.67%. In certain embodiments, the formulation comprises P188 at a concentration in the range of from about 0.3% by weight to about 3% by weight. In certain embodiments, the formulation comprises P188 at a concentration in the range of from about 0.3% by weight to about 2.5% by weight. In certain embodiments, the formulation comprises P188 at a concentration in the range of from about 0.5% by weight to about 2.5% by weight. In certain embodiments, the formulation comprises P188 at a concentration in the range of from about 0.5% by weight to about 2% by weight. In certain embodiments, the formulation comprises P188 at a concentration in the range of from about 0.5% by weight to about 1.5% by weight.

In exemplary embodiments, the formulation comprises P188 at a concentration in the range of from about 0.67% to about 1.67%.

In exemplary embodiments, the formulation comprises P188 at a concentration of about 0.7% (w/w), about 0.8% (w/w), about 0.9% (w/w), about 1.0% (w/w), about 1.1% (w/w), about 1.2% (w/w), about 1.3% (w/w), about 1.4% (w/w), about 1.5% (w/w), about 1.6% (w/w) or about 1.7% (w/w).

In exemplary embodiments, the dry powder formulation comprises about 39% trehalose, about 10.5% leucine, about 2% trileucine, about 8.5% citrate buffer and an active agent.

Exemplary active agents are described throughout, including small molecules, and biologics, such as antibodies and antigen binding fragments thereof.

Suitable sizes for the microparticles of the dry powder formulations are described herein, and in embodiments, the plurality of microparticles have a mass median aerodynamic diameter (MMAD) of about 2 μm to about 4 μm when provided in an aerosol form. Suitable specific surface areas (SSA) of the microparticles are described herein, and include for example, a specific surface area of about 4-7 m²/g. Suitably, the microparticles have an equivalent optical volume mean diameter (oVMD) of about 1 μm to about 5 μm.

In further embodiments, provided herein is a method of preparing a dry powder formulation. In embodiments, the method suitably comprises preparing a liquid feedstock, comprising leucine, about 0.1 mg/mL to about 6 mg/mL trileucine, the active agent, and suitably further comprising a glass stabilization agent. A glass stabilization agent as described herein can be omitted from the dry powder formulations if desired. The liquid feedstock may also comprise a surfactant. The liquid feedstock is prepared by combining these components in a liquid solvent, to create a feedstock in which each of the components is dissolved. Heating may be added as desired or required to increase the solubility of the various components to form the liquid feedstock. Exemplary liquid solvents include water, including deionized water, as well as dilute solutions of alcohols with water. In embodiments, the active agent is suitably added to the liquid feedstock after the addition and dissolution of the remaining components of the feedstock.

In suitable embodiments of the methods of preparation, the leucine and the trileucine are present at a concentration ratio of leucine:trileucine of about 0.1:1 to about 30:1 in the liquid feedstock. As described herein, when preparing a liquid feedstock, the leucine and trileucine are provided as mg/mL amounts. Thus, in such embodiments, the concentration ratio of leucine:trileucine of about 0.1:1 to about 30:1 in the set volume of the liquid feedstock corresponds to the ratio of leucine:trileucine by weight in the liquid feedstock. In further embodiments, the leucine and the trileucine are present at a concentration ratio of leucine:trileucine of about 0.1:1 to about 25:1, about 0.5:1 to about 20:1, about 1:1 to about 20:1, about 1:1 to about 15:1, about 1:1 to about 12:1, about 1:1 to about 10:1, about 1:1 to about 7:1, about 1:1 to about 6:1, or about 1:1:, about 2:1, about 3:1, about 4:1, about 5:1, about 5:1:1:, about 5.2:1 about 5.25:1, about 5.3:1, about 5.4:1, about 5.5:1, about 5.75:1 or about 6:1, in the liquid feedstock.

The liquid feedstock may then be atomized. In certain embodiments, the liquid feedstock is filtered prior to atomizing. In certain embodiments, the liquid feedstock is filtered through a 0.22 micron filter. In certain embodiments, the liquid feedstock comprising leucine and trileucine is filtered prior to the addition of the active agent. In certain embodiments, the liquid feedstock is filtered after the addition of the active agent prior to atomizing. Atomizing refers to converting the liquid feedstock to fine droplets, suitably using a pressurized gas (such as CO₂, or an inert gas). Exemplary devices for producing an atomized liquid feedstock are known in the art and include the use of various atomizing nozzles have desired sizes and flow characteristics. Exemplary parameters for the atomizing including an outlet temperature of about 50° C.-90° C., suitably about 60° C.-80° C., or about 70° C.; a feedstock feed rate of about 8-15 ml/min, suitably about 9-14 ml/min, about 10-13 ml/min, or about 12 ml/min; an atomizer gas flow rate of about 9-15 kg/hour (hr. or h), suitably about 10-14 kg/hr, about 12-14 kg/hr, or about 13 kg/hr; and drying gas flow rate of about 60-100 kg/hr, suitably about 60-90 kg/hr, about 70-90 kg/hr, or about 80 kg/hr.

The atomized liquid feedstock may then be dried, suitably under heat and in combination with flowing air to aid in the drying. The result of the drying yields a plurality of microparticles. Drying temperatures typically range from about 50°−100° C., or about 60°-100° C. or about 70°−90° C.; air flow rate can be on the order of about 10-40 m³/hour.

Exemplary glass stabilization agents, including amorphous saccharides and buffers are described herein, as are suitable amounts of the glass stabilization agents. Suitable amounts of leucine and trileucine are provided throughout as well. As the final, dry powder formulation should contain the recited amounts of leucine and trileucine (and other components), such amounts are also used in the liquid feedstock. The result of the drying process following atomization is that any liquid solvent is removed, and thus the full amount of the original dry weight of the components corresponds to the final dry weight of the compounds in the dry powder formulation. Exemplary active agents are also described herein.

The methods of preparing dry powder formulations described herein suitably provide microparticles having the desired physical characteristics noted, including the desired compressed bulk density, specific surface area and sizes. Exemplary sizes are described herein, as are exemplary SSAs, including a specific surface area of less than about 10 m²/g, suitably about 4-7 m²/g. Suitably the methods provide a plurality of microparticles having an equivalent optical volume mean diameter (oVMD) of about 1 μm to about 5 μm, as described herein; a mass median aerodynamic diameter (MMAD) of about 2 μm to about 4 μm when provided in an aerosol form; a compressed bulk density of about 0.4 g/cm³-0.8 g/cm³.

An additional advantage of the methods of preparing dry powder formulations described herein relates to the high throughput nature of the process. For example, if a flow rate of atomization is set at 20 ml/min, the following throughput in grams/hour, was determined.

TABLE 2
Concentration Implications on Throughput

| | | Max Solids | Throughput |
|---|---|---|---|
| | | Loading | (g/hr) |
| | | (mg/mL) | (at 20 ml/min |
| Leucine | TriLeucine | (Max Solubility | process liq. |
| Content | Content | limited) | flow rate) |
| | 20.0% | 25 | 30 |
| 60.0% | | 33 | 40 |
| 45.0% | | 44 | 53 |
| | 10.0% | 50 | 60 |
| 30.0% | | 67 | 80 |
| 10.5% | 2.0% | 190 | 229 |
| 10.0% | 2.5% | 200 | 240 |
| 8.0% | 2.0% | 250 | 300 |

As set forth, using only trileucine in a feedstock, with a maximum trileucine concentration of 5 mg/mL, a max solids loading of 25 mg/mL was reached (related to the maximum solubility). This results in a throughout of 30 g/hour. With only leucine at 60%, with a maximum leucine concentration of 20 mg/mL a max solids loading of 33 mg/mL was reached, and a throughput of 40 g/hour. Additional results for the use of only leucine and trileucine are also shown. In contrast, for the three feedstocks examined that contained both leucine and trileucine, a maximum solids loading of 250 mg/mL and a throughput of 300 g/hour was reached using only 8% leucine and 2% trileucine. This was a surprising and unexpected finding of the advantages of the methods and formulations disclosed herein, in that a dispersible particle can be provided using relatively small amounts of leucine and trileucine, but also allowing for a large amount of throughput. Such high throughput greatly impacts the ability to scale up production of the dry powder formulations described herein where large amounts of the formulations are required.

The methods and formulations described herein allow for the production of capsules, blister packs, etc., and other suitable containers for dry powder formulations. Such containers can be produced with 10-200 mg of dry powder, suitably 10-100 mg, or 25-75 mg or 50 mg or dry powder formulation. Such containers can suitably deliver 0.1-10 mg of a dry powder formulation to a patient's lungs.

In some embodiments, the use of the methods described herein provide dry powder formulations that can reduce the total number of capsules required for use in an inhalation device. For example, the volume required to deliver 50-100 mg of active agent can be reduced from two larger 00 capsules to a single size 3 capsule.

The methods described herein also provide a mechanism for increasing a compressed bulk density and a specific surface area of a dry powder formulation that comprises a plurality of microparticles. By incorporating leucine and trileucine into the dry powder formulation, a compressed bulk density of about 0.4-1.0 g/cm³ (suitably about 0.5-0.8 g/cm³), can readily be achieved. In addition, a specific surface area of about 5-10 m²/g (suitably about 5 m²/g to about 7 m²/g), can also be achieved. In additional embodiments, the sizes of the microparticles can be formed in the ranges described herein, including microparticles with a mass median aerodynamic diameter (MMAD) of about 2 μm to about 4 μm when provided in an aerosol form.

In embodiments, methods for preparing a dry powder formulation comprising a plurality of microparticles having a compressed bulk density of about 0.4 to about 1.0 g/cm³, are provided, wherein the method comprises incorporating leucine and trileucine into the dry powder formulation at a ratio of leucine:trileucine of about 0.1:1 to about 30:1 by weight.

In further embodiments, methods for preparing a dry powder formulation comprising a plurality of microparticles having a specific surface area of about 5 to about 10 m²/g, are provided, wherein the method comprises incorporating leucine and trileucine into the dry powder formulation at a ratio of leucine:trileucine of about 0.1:1 to about 30:1 by weight.

In still further embodiments, methods for preparing a dry powder formulation comprising a plurality of microparticles, wherein the mass median aerodynamic diameter (MMAD) of the microparticles is about 2 μm to about 4 μm when provided in an aerosol form, are provided, wherein the method comprises incorporating leucine and trileucine into the dry powder formulation at a ratio of leucine:trileucine of about 0.1:1 to about 30:1 by weight.

Suitable amounts of leucine and trileucine are described herein that can be utilized in the methods for increasing a compressed bulk density, providing a specific surface area of a dry powder formulation, and/or providing a specified mass median aerodynamic diameter, include incorporating about 5% to about 15% leucine, and about 1% to about 5% trileucine, suitably incorporating about 8% to about 11% leucine and about 2% to about 4% trileucine. In embodiments, the methods comprising incorporating about 10.5% leucine and about 2% trileucine.

Also provided herein is a method for delivery of a dry powder formulation as described herein to the lungs of a mammalian patient. Suitably, such methods include inhalation of the dry powder formulation in an aerosol form. Mammalian patients that can be administered the dry powder formulations include humans, as well as other mammals such as dogs, cats, sheep, pigs, cows, primates, etc.

Methods for producing an aerosol form of a dry powder formulation are known in the art and include for example, the use of inhaler devices such as a dry-powder inhaler (DPI) (e.g., a Monodose RS01 DPI by PLASTIAPE (Osnago, Italy)). The dry powder formulations described herein can be dispensed into a gas stream by either a passive or an active inhalation device, and remain suspended in the gas for an amount of time sufficient for at least a portion of the microparticles to be inhaled by the patient, so that a portion of the microparticles reaches the lungs.

Also provided herein are methods of treating a medical condition in a mammalian patient, which include administering to the patient by inhalation (including by dry-powder inhaler) the dry powder formulations as described herein.

Medical conditions that can be treated using the methods described herein include those that effect the nervous system, the endocrine system, the muscular system, the cardiovascular system, the digestive system, the respiratory system (and specifically the lungs), hormone systems, the immune system, the reproductive system, etc.

### Additional Exemplary Embodiments

Embodiment 1 is a dry powder formulation including a plurality of microparticles, the microparticles comprising: leucine, about 0.5% to about 10% trileucine by weight, and an active agent, wherein the leucine and the trileucine are present at a ratio of leucine:trileucine of about 0.1:1 to about 30:1 by weight.

Embodiment 2 is a dry powder formulation of embodiment 1, wherein the dry powder formulation has a compressed bulk density of about 0.4 to about 1.0 g/cm3

Embodiment 3 is a dry powder formulation of any preceding embodiment, further comprising a glass stabilization agent.

Embodiment 4 is a dry powder formulation of embodiment 3, wherein the glass stabilization agent is an amorphous saccharide or a buffer.

Embodiment 5 is a dry powder formulation of embodiment 3, wherein the glass stabilization agent comprises an amorphous saccharide and a buffer.

Embodiment 6 is a dry powder formulation of embodiment 4 or embodiment 5, wherein the amorphous saccharide is selected from the group consisting of trehalose, sucrose, raffinose, inulin, dextran, mannitol, and cyclodextrin.

Embodiment 7 is a dry powder formulation of any one of embodiments 4-6, wherein the buffer is selected from the group consisting of a citrate buffer, a phosphate buffer, a histidine buffer, a glycine buffer, an acetate buffer and a tartrate buffer.

Embodiment 8 is a dry powder formulation of any one of embodiments 4-7, wherein the amorphous saccharide is present at about 30% to about 70% by weight.

Embodiment 9 is a dry powder formulation of any one of embodiments 4-8, wherein the amorphous saccharide is trehalose.

Embodiment 10 is a dry powder formulation of embodiment 9, wherein the trehalose is present at about 30%-65% by weight.

Embodiment 11 is a dry powder formulation of any one of embodiments 4-10, wherein the buffer is present at about 1% to about 10% by weight.

Embodiment 12 is a dry powder formulation of any one of embodiments 1-11, wherein the active agent is a small molecule.

Embodiment 13 is a dry powder formulation of any one of embodiments 1-12, wherein the active agent is a biologic.

Embodiment 14 is a dry powder formulation of embodiment 13, wherein the biologic is an antibody or an antigen binding fragment thereof.

Embodiment 15 is a dry powder formulation of any one of embodiments 1-14, wherein the ratio of leucine:trileucine is from about 1:1 to about 12:1 by weight.

Embodiment 16 is a dry powder formulation of any one of embodiments 1-15, wherein the ratio of leucine:trileucine is from about 1:1 to about 7:1 by weight.

Embodiment 17 is a dry powder formulation of any one of embodiments 1-16, wherein the ratio of leucine:trileucine is about 5.25:1 by weight.

Embodiment 18 is a dry powder formulation of any one of embodiments 1-17, comprising about 1% to about 10%, optionally to about 5%, trileucine by weight.

Embodiment 19 is a dry powder formulation of any one of embodiments 1-18, comprising about 8% to about 11% leucine by weight and about 2% to about 4% trileucine by weight.

Embodiment 20 is a dry powder formulation of any one of embodiments 1-19, comprising about 10.5% leucine by weight and about 2% trileucine by weight.

Embodiment 21 is a dry powder formulation of any one of embodiments 1-20, further comprising a surfactant, wherein the surfactant is optionally selected from polysorbate-20 (PS-20), polysorbate-40 (PS-40), polysorbate-60 (PS-60), polysorbate-80 (PS-80) and poloxamer-188.

Embodiment 22 is a dry powder formulation of embodiment 21, wherein the surfactant is PS-80, wherein optionally PS-80 is present at a concentration in the range of from about 0.27% by weight to about 2.7% by weight.

Embodiment 23 is a dry powder formulation of embodiment 22, wherein the PS-80 is present at a concentration of about 1.1% by weight.

Embodiment 23 is a dry powder formulation of embodiment 21, wherein the surfactant is poloxamer-188, wherein optionally poloxamer-188 is present at a concentration in the range of from about 1% by weight to about 10% by weight.

Embodiment 25 is dry powder formulation of claim 23, wherein the poloxamer-188 is present at a concentration in the range of from about 0.67% by weight to about 1.0% by weight.

Embodiment 26 is a dry powder formulation of any one of embodiments 1-25, wherein the plurality of microparticles have an equivalent optical volume mean diameter (oVMD) of about 1 μm to about 5 μm.

Embodiment 27 is a dry powder formulation of any one of embodiments 1-26 wherein the plurality of microparticles have a mass median aerodynamic diameter (MMAD) of about 2 μm to about 4 μm when provided in an aerosol form.

Embodiment 28 is a dry powder formulation of any one of embodiments 2-27, wherein the compressed bulk density is about 0.5 g/cm3 to about 0.8 g/cm3.

Embodiment 29 is a dry powder formulation of any of embodiments 1-28, comprising about 39% trehalose, about 10.5% leucine, about 2% trileucine and about 8.5% citrate buffer.

Embodiment 30 is a dry powder formulation of any of embodiments 1-29, wherein the plurality of microparticles have a specific surface area of less than about 10 m²/g.

Embodiment 31 is a dry powder formulation of embodiment 30, wherein the plurality of microparticles have a specific surface area of from about 4 mg2/g to about 7 m²/g.

Embodiment 32 is a dry powder formulation including a plurality of microparticles, the microparticles comprising about 10.5% leucine by weight, about 2% trileucine by weight, about 8.5% citrate buffer by weight, about 1% to about 40% active agent by weight, about 1.07% by weight polysorbate-80, and trehalose in an amount by weight amount to make up to 100%.

Embodiment 33 is a dry powder formulation of embodiment 32, wherein the active agent is a biologic.

Embodiment 34 is a dry powder formulation of embodiment 33, wherein the biologic is an antibody or an antigen binding fragment thereof.

Embodiment 35 is a method of preparing a dry powder formulation, comprising the steps of: i. preparing a liquid feedstock comprising leucine, about 0.1 mg/mL to about 6 mg/mL trileucine, an active agent and a liquid solvent, wherein the leucine and the trileucine are present at a concentration ratio of leucine:trileucine of about 0.1:1 to about 30:1, ii. atomizing the liquid feedstock and iii. drying the atomized liquid feedstock to form a plurality of microparticles.

Embodiment 36 is a method of embodiment 35, wherein the liquid feedstock further comprises a glass stabilization agent.

Embodiment 37 is a method of embodiment 36, wherein the glass stabilization agent is an amorphous saccharide or a buffer.

Embodiment 38 is a method of embodiment 37, wherein the glass stabilization agent comprises an amorphous saccharide and a buffer.

Embodiment 39 is a method of embodiment 37 or embodiment 38, wherein the amorphous saccharide is selected from the group consisting of trehalose, sucrose, raffinose, inulin, dextran, mannitol, and cyclodextrin.

Embodiment 40 is a method of any one of embodiments 37-39, wherein the buffer is selected from the group consisting of a citrate buffer, a phosphate buffer, a histidine buffer, a glycine buffer, an acetate buffer and a tartrate buffer.

Embodiment 41 is a method of any one of embodiments 39-40, wherein the amorphous saccharide is present at about 30% to about 70%.

Embodiment 42 is a method of any one of embodiments 39-41, wherein the amorphous saccharide is trehalose.

Embodiment 43 is a method of embodiment 42, wherein the trehalose is present at about 30%-65%.

Embodiment 44 is a method of any one of embodiments 37-43, wherein the buffer is present at about 1% to about 10%.

Embodiment 45 is a method of any one of embodiments 35-44, wherein the active agent is a small molecule.

Embodiment 46 is a method of any one of embodiments 35-44, wherein the active agent is a biologic.

Embodiment 47 is a method of embodiment 46, wherein the biologic is an antibody or an antigen binding fragment thereof.

Embodiment 48 is a method of embodiment 35-47, wherein step (a) further comprises adding a surfactant to the liquid feedstock, wherein the surfactant is optionally selected from polysorbate-20 (PS-20), polysorbate-40 (PS-40), polysorbate-60 (PS-60), polysorbate-80 (PS-80) and poloxamer-188.

Embodiment 49 is a method of embodiment 48, wherein the surfactant is PS-80, wherein optionally the PS-80 is present in the liquid feedstock at a concentration in the range of from about 0.02% by weight to about 0.2% by weight.

Embodiment 50 is a method of embodiment 48, wherein the surfactant is poloxamer-188, wherein the poloxamer-188 is present in the liquid feedstock at a concentration in the range of from about 0.75 by weight to about 7.5% by weight.

Embodiment 51 is a method of any one of embodiments 35-450, wherein the concentration ratio of leucine:trileucine in the feedstock is from about 1:1 to about 12:1.

Embodiment 52 is a method of any one of embodiments 35-50, wherein the concentration ratio of leucine:trileucine in the feedstock is from about 1:1 to about 7:1.

Embodiment 53 is a method of any one of embodiments 35-50, wherein the concentration ratio of leucine:trileucine in the feedstock is about 5.25:1.

Embodiment 54 is a method of embodiment 53, wherein the feedstock comprises from about 1 mg/mL to about 1.7 mg/mL trileucine.

Embodiment 55 is a method of any one of embodiments 35-54, wherein the feedstock comprises about 1% to about 5% trileucine.

Embodiment 56 is a method of any one of embodiments 35-55, wherein the feedstock comprises about 8% to about 11% leucine and about 2% to about 4% trileucine.

Embodiment 57 is a method of any one of embodiments 35-56, wherein the feedstock comprises about 10.5% leucine and about 2% trileucine.

Embodiment 58 is a method of any one of embodiments 35-57, wherein the plurality of microparticles have an equivalent optical volume mean diameter (oVMD) of about 1 μm to about 5 μm.

Embodiment 59 is a method of any one of embodiments 35-58, wherein the plurality of microparticles have a mass median aerodynamic diameter (MMAD) of about 2 μm to about 4 μm when provided in an aerosol form.

Embodiment 60 is a method of any one of embodiments 35-59, wherein the plurality of microparticles have a compressed bulk density of about 0.4 g/cm3-0.8 g/cm3.

Embodiment 61 is a method of any of embodiments 35-60, wherein the plurality of microparticles have a specific surface area of less than about 10 m²/g.

Embodiment 62 is a method of embodiment 61, wherein the plurality of microparticles have a specific surface area of about 4 m²/g to about 7 m²/g.

Embodiment 63 is a method of any one of embodiments 35-62 wherein the liquid solvent is water.

Embodiment 64 is a method for preparing a dry powder formulation comprising a plurality of microparticles having a compressed bulk density of about 0.4 to about 1.0 g/cm3, the method comprising incorporating leucine and trileucine into the dry powder formulation at a ratio of leucine:trileucine of about 0.1:1 to about 30:1 by weight.

Embodiment 65 is a method for preparing a dry powder formulation comprising a plurality of microparticles having a specific surface area of about 5 to about 10 m²/g, the method comprising incorporating leucine and trileucine into the dry powder formulation at a ratio of leucine:trileucine of about 0.1:1 to about 30:1 by weight.

Embodiment 66 is a method for preparing a dry powder formulation comprising a plurality of microparticles, wherein the mass median aerodynamic diameter (MMAD) of the microparticles is about 2 μm to about 4 μm when provided in an aerosol form, the method comprising incorporating leucine and trileucine into the dry powder formulation at a ratio of leucine:trileucine of about 0.1:1 to about 30:1 by weight.

Embodiment 67 is a method of any one of embodiments 64-66, comprising incorporating about 1% to about 5% trileucine by weight.

Embodiment 68 is a method of any one of embodiments 64-67, comprising incorporating about 8% to about 11% leucine and about 2% to about 4% trileucine.

Embodiment 69 is a method of any one of embodiments 64-68, comprising incorporating about 10.5% leucine and about 2% trileucine.

Embodiment 70 is a method of any one of embodiments 64-69, wherein the compressed bulk density is from about 0.4 g/cm3 to about 0.8 g/cm3.

Embodiment 71 is a method of any one of embodiments 64-70, wherein the specific surface area is from about 5 m²/g to about 7 m²/g.

Embodiment 72 is a method for delivery of a dry powder formulation to the lungs of a mammalian patient, the method comprising administering to the mammalian patient by inhalation the dry powder formulation of any one of embodiments 1-34, in an aerosol form.

Embodiment 73 is a method of embodiment 72, wherein the dry powder formulation is administered by a dry-powder inhaler (DPI).

Embodiment 74 is a method for treating a medical condition in a mammalian patient, comprising administering to the mammalian patient by inhalation the dry powder formulation of any one of embodiments 1-34, in an aerosol form.

Embodiment 75 is a method of embodiment 74, wherein the dry powder formulation is administered by a dry-powder inhaler (DPI).

Embodiment 76 is a formulation of any one of embodiments 1-34 for use in a method of treatment, wherein the formulation is to be administered by inhalation.

### Examples

### Example 1: Evaluating Physical Characteristics of Spray-Dried Formulations Comprising Leucine and Trileucine

The following methods evaluate the impact of trileucine and leucine ratios in dry powder formulations on particle properties.

In total, 24 powders of varying trileucine, leucine, and trehalose (TLT) wt % were spray-dried on a pilot scale spray dryer using identical process parameters at a total feedstock solids concentration of 10%. Since feedstocks were prepared at a total solids concentration of 10% (100 mg/mL), all wt % values in this study are also identical to concentration values (mg/mL). The range of concentration values for each particle excipient is shown in Table 3.

TABLE 3
Particle Component Composition Ranges

| | Minimum | Maximum |
|---|---|---|
| Component | Value | Value |
| TriLeucine | 0.71 mg/mL | 5.72 mg/mL |
| Leucine | 0.62 mg/mL | 19.94 mg/mL |
| Trehalose | 65.84 mg/mL | 90.16 mg/mL |
| TriSodium Citrate | 8.5 mg/mL | 8.5 mg/mL |

Each feedstock (Table 4) was prepared by dissolving the excipients in water. Once all excipients were fully dissolved, feedstocks were spray dried, using the following process parameters: outlet temperature, 70° C.; feedstock feed rate, 12 ml/min; atomizer gas flow, 13 kg/hr; and drying gas flow, 80 kg/hr. The parameters were selected to achieve particle and aerosol properties for a dry powder formulation intended for inhalation. Each of the 24 formulations were manufactured at an 18 g batch-size to provide sufficient powder for characterization and product performance evaluation. Batches were randomized and produced across two days.

TABLE 4
Feedstock Concentrations for Formulations 1-24

| | | | | TriSodium | leucine/ |
|---|---|---|---|---|---|
| | Trileucine | Leucine | Trehalose | Citrate | trileucine |
| | Conc. | Conc. | Conc. | Conc | concentration |
| Run | mg/mL | mg/mL | mg/mL | mg/mL | ration |
| 1 | 1.43 | 13.08 | 76.99 | 8.5 | 9.1 |
| 2 | 0.71 | 0.62 | 90.16 | 8.5 | 0.9 |
| 3 | 0.71 | 4.98 | 85.80 | 8.5 | 7.0 |
| 4 | 0.71 | 19.94 | 70.85 | 8.5 | 28.1 |
| 5 | 1.43 | 16.20 | 73.87 | 8.5 | 11.3 |
| 6 | 2.86 | 14.95 | 73.69 | 8.5 | 5.2 |
| 7 | 2.86 | 9.97 | 78.67 | 8.5 | 3.5 |
| 8 | 2.86 | 19.94 | 68.70 | 8.5 | 7.0 |
| 9 | 2.86 | 4.36 | 84.28 | 8.5 | 1.5 |
| 10 | 5.72 | 19.94 | 65.84 | 8.5 | 3.5 |
| 11 | 0.71 | 15.58 | 75.21 | 8.5 | 21.9 |
| 12 | 5.72 | 15.58 | 70.20 | 8.5 | 2.7 |
| 13 | 5.00 | 11.84 | 74.66 | 8.5 | 2.4 |
| 14 | 0.71 | 7.48 | 83.31 | 8.5 | 10.5 |
| 15 | 2.86 | 0.62 | 88.02 | 8.5 | 0.2 |
| 16 | 3.57 | 18.07 | 69.86 | 8.5 | 5.1 |
| 17 | 5.72 | 0.62 | 85.16 | 8.5 | 0.1 |
| 18 | 5.72 | 6.23 | 79.55 | 8.5 | 1.1 |
| 19 | 1.43 | 11.22 | 78.85 | 8.5 | 7.0 |
| 20 | 0.71 | 9.97 | 80.82 | 8.5 | 14.0 |
| 21 | 4.29 | 3.12 | 84.10 | 8.5 | 0.7 |
| 22 | 4.29 | 16.82 | 70.39 | 8.5 | 3.9 |
| 23 | 5.72 | 9.97 | 75.81 | 8.5 | 1.7 |
| 24 | 3.57 | 9.97 | 77.96 | 8.5 | 2.8 |

The following physical powder characteristics were tested for all formulations

TABLE 5
Particle Parameters Analyzed

| Analysis/DOE Output | Instrument |
|---|---|
| Residual Moisture Content | Oven KF |
| Primary Particle Size Distribution | Sympatec R |
| Glass Transition Temperature | DSC |
| (Tg) | |
| Compressed Bulk Density¹ | GeoPyc |
| SEM Visual Morphology | SEM |
| (qualitative) | |
| Specific Surface Area | BET |
| Crystallinity (qualitative) | XRPD |
| Excipient Surface Coverage on | ToF-SIMS |
| Particle | |

Compression force of 300,000 N/m², or 38N if using a 12.7 mm sample chamber

The compressed bulk density (CBD) of the powders were measured using a GeoPyc® Model 1360 density analyzer (Micromeritics, Norcross, Ga.). Powder samples were prepared in a low humidity environment (<5% RH), before transfer into the density analyzer sample chamber that had been purged with nitrogen gas. The net weight of the powder sample was recorded, and then a compression force of 12N was applied to the sample by a plunger, at a rate of 300 consolidation steps per second. The linear distance travelled by the plunger for each consolidation step was translated into a volume displacement of the powder sample. An average of the measurements from each consolidation step was then transformed into a calculated bulk density value, expressed in g/cm³.

The results show that the leucine and trileucine content were found to have a significant impact on particle properties. Trileucine was identified as being the primary factor with the largest impact, while leucine was identified as a secondary factor with also a notable impact. The results are summarized in Table 6.

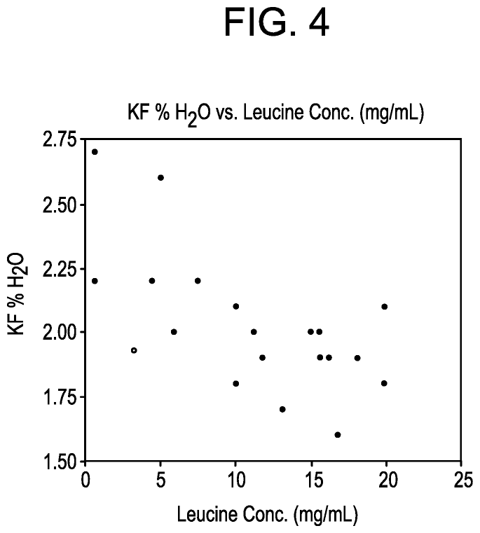

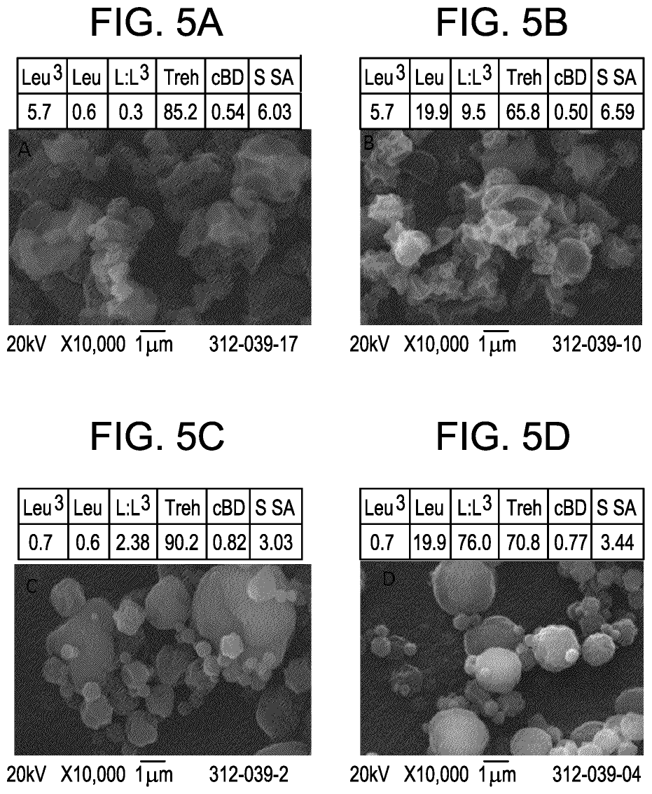

TABLE 6
Results of Particle Characterization

| Analysis/DOE Output | Impact |
|---|---|
| Residual Moisture Content | Correlation of reducing moisture content with increasing |
| | Leucine Content (see FIG. 4) |
| Primary Particle Size | No trend observed with Leucine or TriLeucine |
| Distribution | |
| Glass Transition Temperature | No trend observed with Leucine or TriLeucine |
| (Tg) | |
| Compressed Bulk Density¹ | Primary Negative Correlation with TriLeucine; Secondary |
| | Negative Correlation with Leucine. cBD of between 0.45 |
| | to 0.85 g/cm³ (FIG. 2A) |
| SEM Visual Morphology | Positive Correlation with TriLeucine and surface |
| | roughness/rugosity (FIG. 5A-5D) |
| Specific Surface Area | Primary Positive Correlation with TriLeucine; Secondary |
| | Positive Correlation with Leucine The SSA of the |
| | formulations is from 2.5 to 6.5 m²/g (FIG. 3) |
| Crystallinity | Greater Crystallinity was achieved at Higher Leucine |
| | contents combined with lower TriLeucine contents |
| Excipient Particle Surface | Greater Surface coverage achieved with increasing |
| Coverage | TriLeucine and Leucine wt % (50% particle coverage of |
| | Trileucine was achieved above 1.4 wt %) |

Compression force of 300,000 N/m², or 38N if using a 12.7 mm sample chamber.

### Example 2— Aerosol Performance Characteristics of Leucine/Trileucine Formulations

The following example evaluates the aerosol performance of formulations comprising leucine and trileucine in a dry powder inhaler device. The aerosol performance outputs listed in Table 7 were tested on 20 of the 24 formulations listed in Table 4. All product performance characterization was completed using a Monodose RS01 device, with size 3 capsules. Next Generation Impactor (NGI) analysis was performed at a 60 L/min flow rate.

Cascade impaction testing was performed as per USP <601> to measure the aerosol performance of the spray dried formulations when delivered from a dry powder inhaler device. The cascade impactor apparatus used was the Next Generation Impactor (NGI; USP41, Chapter <601>). For the aerosol measurements made in these examples, one Size 3 HPMC capsule containing the spray dried powder formulation was dispersed from the dry powder inhaler device and delivered into the NGI under a vacuum pulled at 60 L/min as per USP methodology. Samples from each stage of the NGI were recovered and assayed for protein content by UV absorption at 280 nm. The main aerosol performance parameters calculated from these measurements were a) Fine Particle Fraction <5 μm (FPF<5 μm), defined as the fraction of powder emitted from the device that is measured to be <5 μm in aerodynamic particle diameter; and b) median mass aerodynamic diameter MMAD.

TABLE 7
Aerosol Characterization

| | Instrument/ |
|---|---|
| | Technique |
| Analysis/DOE Output | used |
| Mean mass aerodynamic | NGI |
| diameter (MMAD) | |
| % Device Deposition | NGI |
| % FPF <5 um | NGI |

The results of the aerosol analysis are summarized in Table 8.

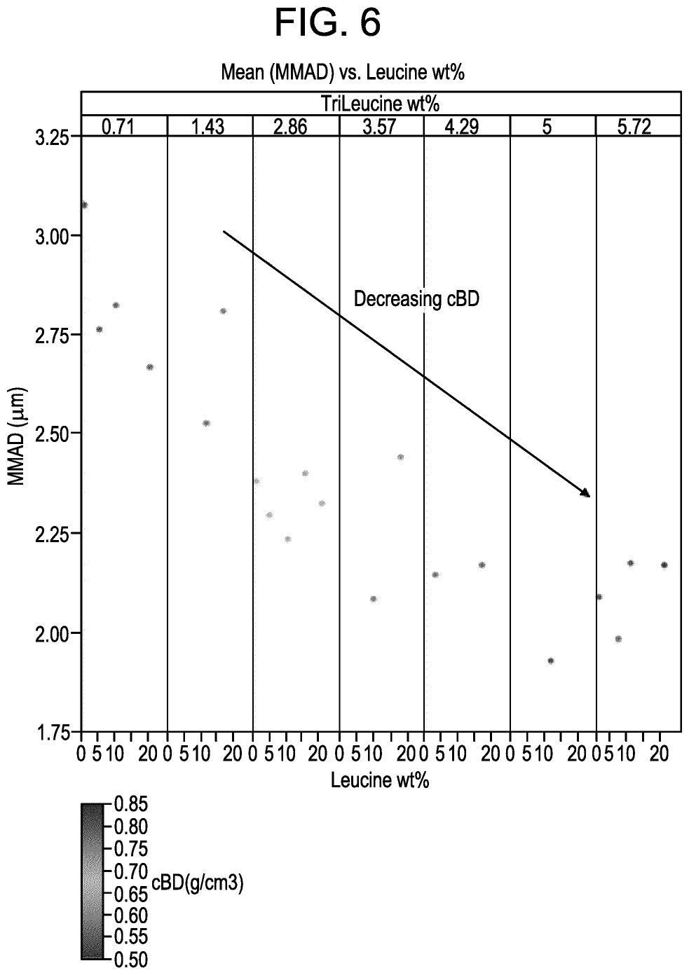

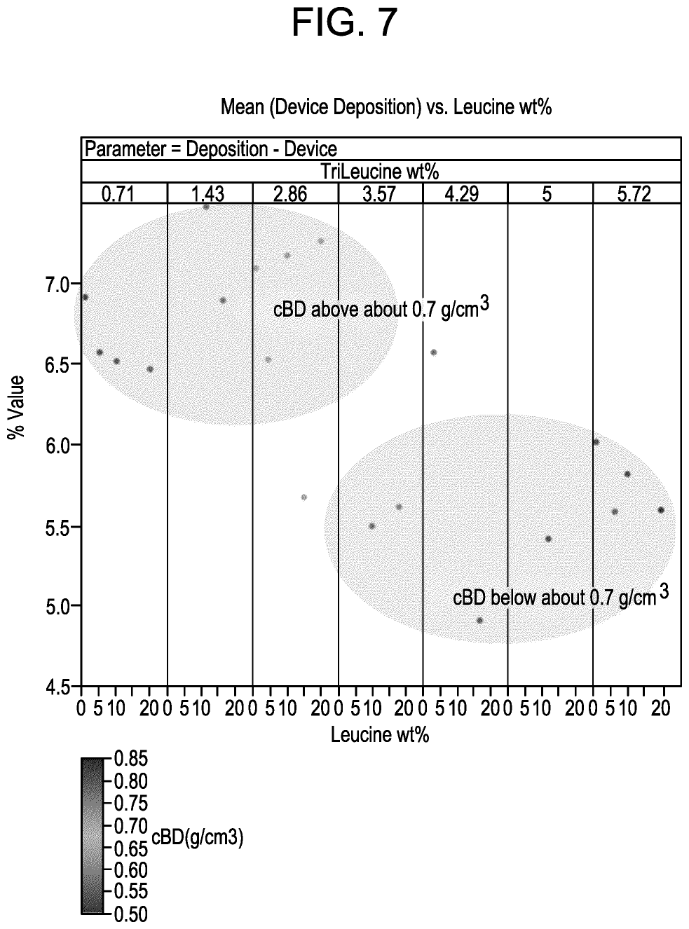

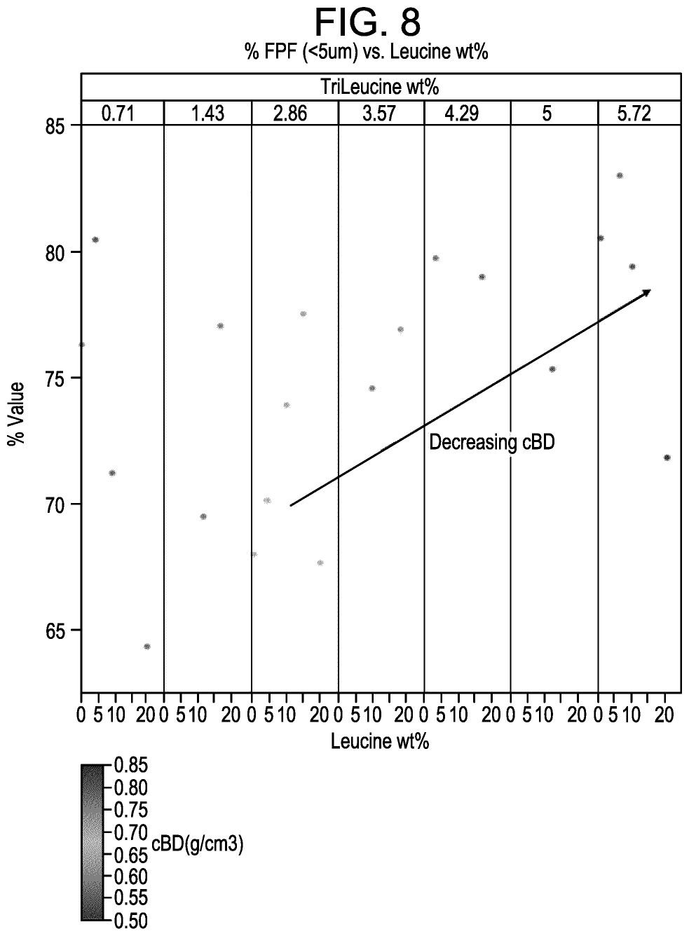

TABLE 8
Results of Aerosol Characterization

| Analysi/DOE Output | Impact |
|---|---|
| MMAD (median mass | Strong Negative Correlation with TriLeucine. |
| aerodynamic diameter) | A range of MMAD values of from 1.75 to 3.25 μm |
| | were achieved (FIG. 6) |
| % Device Deposition | Stepwise correlation with TriLeucine. In general, a |
| | trileucine wt % of above 3% resulted in a reduction |
| | in device deposition (FIG. 7). |
| % FPF (fine particle fraction) | Positive Correlation with TriLeucine |
| <5 um | Negative Correlation with Leucine. All 20 of the |
| | tested formulations had FPFs of >60%, indicating |
| | good performance (FIG. 8). |

### Example 3— Saturation Rate Modelling

To understand the crystallinity results of Example 1, modelling was carried out to evaluate the rate of saturation of leucine and trileucine during the expected particle formation process in the spray dryer. This modelling identified that leucine crystallization at the surface was largely dependent on the concentration ratio of the two excipients.

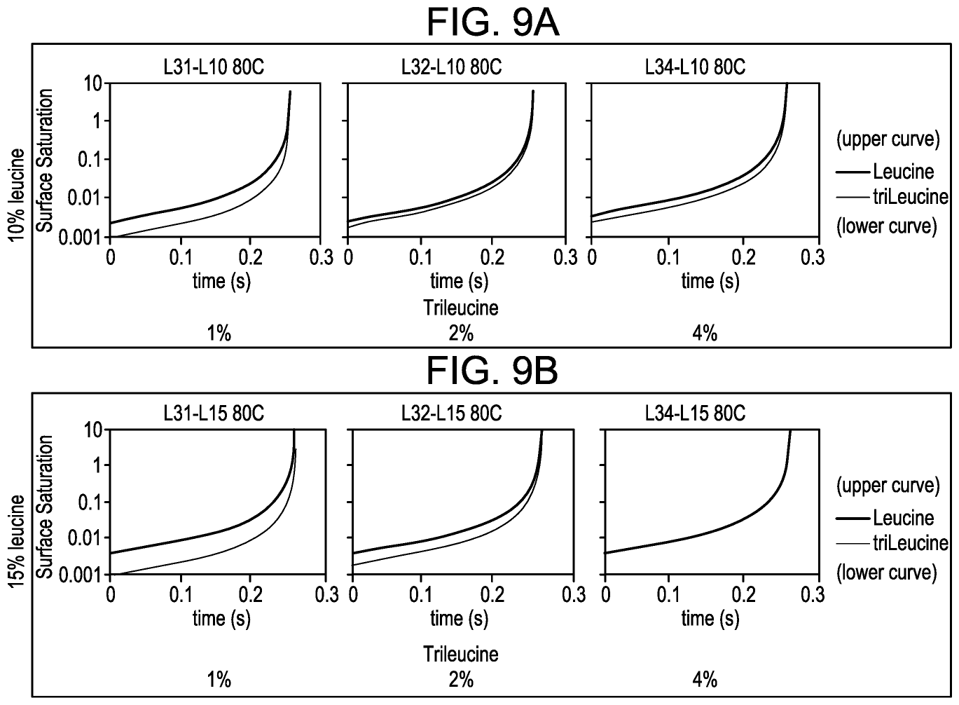

At the leucine:trileucine concentration ratio of 3.8, saturation curves for both excipients overlap resulting in co-saturation during particle formation (FIGS. 9A-9B). This inhibits leucine crystallization even when leucine is present in high concentrations. Particle formulations that had high leucine concentrations of approximately >15 mg/mL and low trileucine concentrations of approximately <1 mg/mL saw increased particle crystallinity.

### Example 4— Assessing the Impact of Feedstock Factors on the Physical Characteristics of Leucine/Trileucine Formulations

A second set of leucine:trileucine formulations were generated to evaluate the impact of buffer wt %, evaluate the impact of total leucine: trileucine wt % across varying feedstocks concentrations, and evaluate the impact of leucine/trileucine varying concentrations at the set ratio of 3.8 (leucine:trileucine).

In total, 27 powders varying in trileucine, leucine, trehalose, and trehalose wt % were generated. Three Factors were evaluated at three levels to create a full-factorial study design outlined in Table 9. The three factors were Citrate buffer wt %, Total Feedstock Concentration, and total trileucine and leucine concentration. All formulations were held at a constant leucine to trileucine ratio of 3.8.

TABLE 9
Particle composition ranges tested in Example 4.

| | Minimum | Mid | Maximum |
|---|---|---|---|
| Component | Level | Level | Level |
| Citrate Buffer | 4.25 wt % | 8.5 wt % | 12.75 wt % |
| Feedstock Conc. | 50 mg/mL | 100 mg/mL | 200 mg/mL |
| Total TriLeu/Leu | 6.3 mg/mL | 12.6 mg/mL | 18.9 mg/mL |
| concentration | | | |
| Trileucine | 1.3 mg/mL | 2.6 mg/mL | 3.9 mg/mL |
| Leucine | 5.0 mg/mL | 10 mg/mL | 15 mg/mL |

The 27 feedstocks that were used are shown in Table 10. Each feedstock was prepared by dissolving the excipients trehalose, trisodium citrate, leucine and trileucine in water. Once all excipients were fully dissolved feedstocks were spray dried, using the following process parameters: outlet temperature, 70° C.; feedstock feed rate, 18 ml/min; atomizer gas flow, 13 kg/h; and drying gas flow, 155 kg/hr. These parameters were selected to achieve the target particle and aerosol properties for a dry powder formulation intended for inhalation. Each of the 27 formulations were manufactured at 65 g batch sizes to provide ample powder for characterization. Batches were randomized and produced across several days.

TABLE 10
Compositions of Formulations
Evaluated in Example 4

| | | Leucine + | |
|---|---|---|---|
| | Feedstock | TriLeucine | Citrate |
| | Conc. | Conc. | Buffer |
| Run | mg/mL | mg/mL | Wt % |
| 1 | 50 | 6.3 | 4.25 |
| 2 | 50 | 6.3 | 8.50 |
| 3 | 50 | 6.3 | 12.75 |
| 4 | 50 | 12.6 | 4.25 |
| 5 | 50 | 12.6 | 8.50 |
| 6 | 50 | 12.6 | 12.75 |
| 7 | 50 | 18.9 | 4.25 |
| 8 | 50 | 18.9 | 8.50 |
| 9 | 50 | 18.9 | 12.75 |
| 10 | 100 | 6.3 | 4.25 |
| 11 | 100 | 6.3 | 8.50 |
| 12 | 100 | 6.3 | 12.75 |
| 13 | 100 | 12.6 | 4.25 |
| 14 | 100 | 12.6 | 8.50 |
| 15 | 100 | 12.6 | 12.75 |
| 16 | 100 | 18.9 | 4.25 |
| 17 | 100 | 18.9 | 8.50 |
| 18 | 100 | 18.9 | 12.75 |
| 19 | 200 | 6.3 | 4.25 |
| 20 | 200 | 6.3 | 8.50 |
| 21 | 200 | 6.3 | 12.75 |
| 22 | 200 | 12.6 | 4.25 |
| 23 | 200 | 12.6 | 8.50 |
| 24 | 200 | 12.6 | 12.75 |
| 25 | 200 | 18.9 | 4.25 |
| 26 | 200 | 18.9 | 8.50 |
| 27 | 200 | 18.9 | 12.75 |

The following physical powder characteristics were tested for all 27 formulations:

TABLE 11
Particle Parameters Analyzed

| Analysis/DOE Output | Instrument |
|---|---|
| Primary Particle Size Distribution | Sympalec R |
| Residual Water | Oven KF |
| SEM Visual Morphology | SEM |
| (qualitative) | |
| Surface Area | SSA-BET |
| Compressed Bulk Density¹ | GeoPyc |
| Glass Transition Temperature | DSC |
| (Tg) | |
| Aerodynamic Particle Size | aVMD |
| Moisture Sorption | SMS DVS |
| Crystallinity | XRPD |
| Surface Coverage | ToF-SIMS |

Compression force of 300,000 N/m², or 38N if using a 12.7 mm sample chamber.

The results show that leucine and trileucine total concentration and wt % have the most significant impact on particle properties, whereas feedstock concentration appeared to be a secondary factor. The results are summarized in Table 12.

TABLE 12
impact of feedstock concentration, leucine and trileucine wt % and
concentration on particle characteristics

| Analysis/DOE Output | Impact |
|---|---|
| Primary Particle Size | Positive correlation with feedstock concentration |
| Distribution | |
| Residual Water | Negative Correlation with total leucine and trileucine wt % |
| | Weak positive correlation with Citrate Buffer wt % |
| Surface Area | Positive Correlation with total leucine and trileucine |
| | Negative Correlation with increasing Feedstock Concentration |
| Compressed Bulk | Negative Correlation with total leucine and trileucine wt % |
| Density¹ | Negative Correlation with total leucine and trileucine |
| | concentration |
| Glass Transition | Positive Correlation with total leucine and trileucine wt % |
| Temperature (Tg) | |
| Crystallinity | No major trend |

### Example 5— Generating Inhalable Leucine/Trileucine Formulations Comprising an Anti-Interleukin-4 Antibody Binding Fragment (Fab)

The following example examines whether a biologic active ingredient could be included in the leucine/trileucine formulation system described above to create a product suitable for inhalable delivery. Described herein is the use of an anti-interleukin-4 antibody binding fragment (Fab), the variable antigen-binding region of an IgG1 monoclonal that binds specifically to IL-4. The anti-IL-4 Fab was formulated as a spray-dried powder for inhaled delivery according to the mass concentrations outlined in Table 13.

TABLE 13
Compositions of formulation comprising anti-IL-4 Fab

| | anti-IL-4 | | | | Citrate, |
|---|---|---|---|---|---|
| | Fab | Trehalose | Leucine | Trileucine | pH 6.0 |
| Formulation | [% w/w] | [% w/w] | [% w/w] | [% w/w] | [% w/w] |
| #1 | 14.5 | 64.5 | 10.5 | 2.0 | 8.5 |
| #2 | 40.0 | 39.0 | 10.5 | 2.0 | 8.5 |

The anti-IL-4 fab was initially in a liquid formulation at a concentration of approximately 50 mg/mL, in 105 mM trehalose, 30 mM citrate, pH 6.0. Leucine, trileucine, trehalose and citrate were dissolved into a separate aqueous solution, which was then added to the anti-IL-4 Fab solution to create bulk liquid feedstock solutions for spray drying. Table 14 summarizes the feedstock compositions prepared to generate the target powder formulation compositions. The liquid feedstock solutions were then spray dried, using the following process parameters: outlet temperature, 70° C.; feedstock feed rate, 12 ml/min; atomizer gas flow, 13 kg/hr; and drying gas flow, 130 kg/hr, which were selected to achieve the target particle and aerosol properties for a dry powder formulation intended for inhalation.

TABLE 14
Compositions of liquid feedstocks for spray drying

| | Formulation | Formulation |
|---|---|---|
| | #1 | #2 |
| Anti-IL-4 Fab [mg/mL] | 8.7 | 24.0 |
| Trehalose [mg/mL] | 38.7 | 23.4 |
| Leucine [mg/mL] | 6.3 | 6.3 |
| Trileucine [mg/mL] | 1.2 | 1.2 |
| Citrate pH 6.0 [mg/mL] | 5.1 | 5.1 |
| Total feedstock | 60.0 | 60.0 |
| concentration [mg/mL] | | |

Results from powder and aerosol performance characterization of the spray dried formulations are summarized in Table 15. Powder and aerosol characterization were performed according to the specified methodologies. Aerosol performance of the 14.5% and 40% w/w anti-IL-4 Fab spray dried formulation were tested with 65 mg and 50 mg powder, filled in a size 3 HPMC capsule, and dispersed from a dry powder inhaler device.

TABLE 15
Powder and aerosol properties of anti-IL-4 Fab formulations

| | Formulation | Formulation |
|---|---|---|
| | #1 | #2 |
| oVMD [μm] (n = 2) | 1.5 (d50) | 1.7 (d50) |
| | 3.4 (d90) | 3.6 (d90) |
| CBD [g/cm³] (n = 2) | — | 0.59 |
| SSA [m²/g] (n = 2) | — | 4.52 |
| FPM_{<5μm} [mg anti-IL4 Fab] (n = 3) | 4.9 | 11.6 |
| FPF_{<5μm} [%] (n = 3) | 63 | 68 |
| MMAD (n = 3) | 3.1 | 3.0 |

The results demonstrate that spray dried formulations containing leucine and trileucine in a ratio of 5.25:1 were effective in achieving the target powder properties for two distinct doses of anti-IL-4 Fab. High powder CBD (0.59 g/cm³) was observed, as well as low specific surface area (4.52 m²/g), while also achieving very high powder dispersibility (MMAD 3.0-3.1 μm; FPF<5 μm of about 60-70-%).

Example 6— Generating inhalable leucine/trileucine formulations comprising an anti-TSLP antibody binding fragment (Fab)

The characteristics of another formulation comprising a different Fab were tested. An anti-TSLP Fab was used, derived from a human IgG1 monoclonal antibody that specifically binds TSLP (thymic stromal lymphopoetin) (see the sequences set forth in SEQ ID NOS: 1-8 provided herein). Distinct formulations comprising the mass concentrations outlined in Table 16 were generated.

TABLE 16
Compositions of spray dried formulations containing anti-TSLP Fab

| | Anti-TSLP | | | | Citrate, |
|---|---|---|---|---|---|
| | Fab | Trehalose | Leucine | Trileucine | pH 6.0 |
| Formulation | [% w/w] | [% w/w] | [% w/w] | [% w/w] | [% w/w] |
| #1 | 1 | 78 | 10.5 | 2 | 8.5 |
| #2 | 12 | 67 | 10.5 | 2 | 8.5 |
| #3 | 40 | 39 | 10.5 | 2 | 8.5 |

The anti-TSLP Fab was initially received in a liquid buffer comprising 105 mM trehalose, 30 mM citrate, pH 6.0. Leucine, trileucine, trehalose and citrate were dissolved into a separate aqueous solution, which was then added to the anti-TSLP Fab solution to create bulk liquid feedstock solutions for spray drying. Table 17 summarizes the feedstock compositions prepared in order to achieve the target powder formulation compositions. The liquid feedstock solutions were then spray dried, using process parameters listed in Table 18. The parameters were selected to achieve the target particle and aerosol properties for a dry powder formulation intended for inhalation.

TABLE 17
Compositions of Liquid Feedstocks for Spray Drying

| | Formulation | Formulation | Formulation |
|---|---|---|---|
| | #1 | #2 | #3 |
| Anti-TSLP Fab [mg/mL] | 0.75 | 9.0 | 24 |
| Trehalose [mg/mL] | 58.5 | 50.3 | 23.4 |
| Leucine [mg/mL] | 7.9 | 7.9 | 6.3 |
| Trileucine [mg/mL] | 1.5 | 1.5 | 1.2 |
| Citrate pH 6.0 [mg/mL] | 6.4 | 6.4 | 5.1 |
| Total feedstock | 75 | 75 | 60 |
| concentration [mg/mL] | | | |

TABLE 18
Key spray drying process parameters

| | Formulation | Formulation | Formulation |
|---|---|---|---|
| | #1 | #2 | #3 |
| Outlet temperature (° C.) | 70 | 70 | 70 |
| Feedstock feed rate (mL/min) | 20 | 17 | 3 |
| Atomizer Gas Flow (kg/h) | 13 | 13 | 2.1 |
| Drying gas flow (kg/hr) | 155 | 155 | 59.5 |

Results from powder and aerosol performance characterization of the spray dried formulations are summarized in Table 19. For aerosol performance measurements, all three formulations were tested with 20 mg of spray dried powder filled in a Size 3 HPMC capsule and dispersed from a dry powder inhaler device.

TABLE 19
Powder and aerosol properties of spray dried anti-TSLP
Fab-containing formulations

| | Formulation #1 | Formulation #2 | Formulation #3 |
|---|---|---|---|
| | 1% w/w | 12% w/w | 40% w/w |
| | anti-TSLP Fab | anti-TSLPF ab | anti-TSLP Fab |
| oVMD [μm] (n = 2) | 1.5 (d50) | 1.5 (d50) | 1.9 (d50) |
| | 3.5 (d90) | 3.5 (d90) | 4.1 (d90) |
| cBD [g/cm³] | 0.72 | 0.70 | 0.58 |
| SSA [m²/g] | 3.5 | 4.12 | 4.6 |
| FPM_{<5μm} [mg anti- | 0.17 | 1.9 | 5.9 |
| TSLP Fab] (n = 3) | | | |
| FPF_{<5μm} [%] (n = 3) | 95.1 | 94.3 | 85.4 |
| MMAD (n = 3) | 2.3 | 2.2 | 2.7 |

Of particular note is the success in filling 50 mg of Formulation #3 into a single Size 3 HPMC capsule, attributable to the high bulk density of the powder. The high compressed bulk density (CBD) enabled the delivery of a very high payload from a single capsule (FPM <5 μm of about 14 mg, FPF of 82%, MMAD of 2.4 μm).

In addition, Formulation #3, exhibits a similar CBD (0.58 g/cm3) and SSA (4.6 m²/g) to that of anti-IL-4 Fab Formulation #2 (cBD=0.59 g/cm3, SSA=4.5 m²/g), suggesting that the powder properties translate between pharmaceutical formulations comprising different active ingredients from the same class of molecule.

### Example 7— Powder and Aerosol Properties of Spray Dried Anti-Tslp Formulations at Three Batch Sizes

This example provides an analysis of the powder and aerosol properties of the anti-TSLP Fab leucine/trileucine formulations using greater batch sizes to enable non-GLP and GLP inhalation toxicology studies. Scale up requires the use of alternative scale spray dryer equipment, and adjustments to spray drying process parameters, to account for increased heat and mass flow through the system and the need for extended processing runs.

Three batches of a spray dried anti-TSLP Fab formulations were manufactured in increasing batch sizes. The batches comprised: anti-TSLP Fab 40% w/w, trehalose 39% w/w, leucine 10.5% w/w, trileucine 2% w/w, and citrate pH 6.0 8.5% w/w. The process parameters selected for each batch are shown in Table 20.

TABLE 20
Spray dryer process parameters for three anti-TSLP Fab formulation batches
of increasing batch size

| | Batch #1 | Batch #2 | Batch #3 |
|---|---|---|---|
| Feedstock concentration [mg/mL] | 60 | 75 | 75 |
| Outlet temperature (° C.) | 70 | 70 | 70 |
| Feedstock feed rate (mL/min) | 3 | 5 | 12 |
| Atomizer Gas Flow (kg/h) | 2.1 | 2.1 | 13.3 |
| Drying gas flow (kg/hr) | 59.5 | 59.5 | 155 |
| Total batch size* | 8.5 g | 348 g | 1.2 kg |
| Spray dryer | Lab-scale | Lab-scale | Intermediate- |
| | | | scale |
| Days/hours of production | 1 day/1.1 h | 2 days/15.9 h | 2 days/22.4 h |

*Processed powder weight.

Aerosol performance testing of Batch #1 was performed with a powder fill mass of 50 mg in a Size 3 HPMC capsule, while Batches #2 and #3 were tested with a 20 mg fill mass. There is a slight increase in the oVMD as the batch size increased from batch size 8.5 g to 1.2 kg. A compressed bulk powder density (cBD) of between 0.45 and 0.85 g/cm³ was achieved for Batch 3. The aerosol performance of the powders were also maintained independent of batch size, with a high payload delivery of anti-TSLP Fab from the capsule-based inhaler device. The demonstrates the scalability of the formulation with minimal adjustments to the spray dryer process. The full results of powder characterization and aerosol performance testing are summarized in Table 21.

TABLE 21
Powder properties and aerosol performance for three anti-TSLP
Fab batches of increasing batch size

| | Batch #1 | Batch #2 | Batch #3 |
|---|---|---|---|
| oVMD [μm] (n = 2) | 1.5 (d50) | 1.7 (d50) | 1.9 (d50) |
| | 3.2 (d90) | 3.8 (d90) | 4.1 (d90) |
| CBD [g/cm³] | — | — | 0.58 |
| SSA [m²/g] | — | — | 4.6 |
| FPM_{<5μm} [mg anti- | 14.3 | 5.6 | 5.9 |
| TSLP Fab] (n = 3) | | | |
| FPF_{<5μm} [%] (n = 3) | 81.9 | 83.4 | 85.4 |
| MMAD [μm] (n = 3) | 2.4 | 2.4 | 2.7 |

### Example 8 Further Characterization of Leucine/Trileucine Formulations Comprising a Surfactant

Additional batches of trileucine/leucine formulations comprising varying amounts of PS-80 were generated. The formulation compositions and process parameters for the generation of each batch are shown in Table 22. Otherwise, formulation generation was as described in example 6.

TABLE 22
Formulation compositions and spray dry process parameters
for formulations comprising increasing amounts of PS-80

| | | | 40% FAB₁, | | 40% FAB₁, | | 40% FAB₁, | | 40% FAB₁, | | 40% FAB₁, | |
|---|---|---|---|---|---|---|---|---|---|---|---|---|
| | 40% FAB₁, | | 0.27% w/w | | 0.67% w/w | | 1.33% w/w | | 2.00% w/w | | 2.67% w/w | |
| | control | | PS80 (0.02% | | PS80 (0.05% | | PS80 (0.10% | | PS80 (0.15% | | PS80 (0.20% | |
| Description | (no PS80) | | w/v PS80) | | w/v PS80) | | w/v PS80) | | w/v PS80) | | w/v PS80) | |
| Composition | % w/w | mg/ml | % w/w | mg/ml | % w/w | mg/ml | % w/w | mg/ml | % w/w | mg/ml | % w/w | mg/ml |
| FAB₁ | 40.00 | 29.60 | 40.00 | 29.60 | 40.00 | 30.00 | 40.00 | 30.00 | 40.00 | 30.00 | 40.00 | 29.20 |
| TriSoduim | 7.75 | 5.74 | 7.75 | 5.74 | 7.75 | 5.81 | 7.75 | 5.81 | 7.75 | 5.81 | 7.75 | 5.66 |
| Citrate | | | | | | | | | | | | |
| Anhydrous | | | | | | | | | | | | |
| Citric Acid | 0.75 | 0.56 | 0.75 | 0.56 | 0.75 | 0.56 | 0.75 | 0.56 | 0.75 | 0.56 | 0.75 | 0.55 |
| Anhydrous | | | | | | | | | | | | |
| Trehalose | 39.00 | 28.86 | 38.73 | 28.66 | 38.34 | 28.75 | 37.67 | 28.26 | 37.02 | 27.76 | 36.29 | 26.49 |
| Anhydrous | | | | | | | | | | | | |
| TriLeucine | 2.00 | 1.48 | 2.00 | 1.48 | 2.00 | 1.50 | 2.00 | 1.50 | 2.00 | 1.50 | 2.00 | 1.46 |
| Leucine | 10.50 | 7.77 | 10.50 | 7.77 | 10.50 | 7.88 | 10.50 | 7.88 | 10.50 | 7.88 | 10.50 | 7.67 |
| PS80 | 0.00 | 0.00 | 0.27 | 0.20 | 0.66 | 0.50 | 1.33 | 0.99 | 1.98 | 1.49 | 2.71 | 1.98 |

| | | | | | | |
|---|---|---|---|---|---|---|
| Drying | 850 | 850 | 850 | 850 | 850 | 850 |
| gas (slpm) | | | | | | |
| Liq feed | 5 | 5 | 5 | 5 | 5 | 5 |
| rate (ml/min) | | | | | | |
| Atomizer (slpm) | 30 | 30 | 30 | 30 | 30 | 30 |
| Inlet temp (° C.) | 100 | 100 | 100 | 100 | 100 | 100 |
| Outlet temp (° C.) | 70 | 70 | 70 | 70 | 70 | 70 |
| GLR | 7 | 7 | 7 | 7 | 7 | 7 |
| Run time (hr) | 0.68 | 0.27 | 0.67 | 0.67 | 0.67 | 0.68 |

The aerosol properties of the formulations in Table 22 were analyzed using the methods disclosed in Example 7. The results of the analysis are shown in Table 23.

TABLE 23
aerosol performance of formulations comprising PS-80

| | | FPM | |
|---|---|---|---|
| | % FPF | (<5.0 um) | MMAD |
| Description | (<5.0 um) | (mg) | (um) |
| 40% FAB₁, control | 78 | 5.5 | 2.55 |
| 40% FAB₁, | 77 | 5.9 | 2.56 |
| 0.27% w/w PS80 | | | |
| 40% FAB₁, | 70 | 4.4 | 2.68 |
| 0.67% w/w PS80 | | | |
| 40% FAB₁, | 75 | 4.5 | 2.7 |
| 1.33% w/w PS80 | | | |
| 40% FAB₁, | 82 | 4.2 | 2.27 |
| 2.00% w/w PS80 | | | |
| 40% FAB₁, | 68 | 4.0 | 2.63 |
| 2.67% w/w PS80 | | | |

Aggregate content, oVMD, residual moisture content, Tg, cBD and SSA were also measured using the methods described in the preceding examples. Results of the powder property analysis are shown in Table 24.

TABLE 24
Powder properties of dry powder formulations comprising
FAB₁ and varying (w/w) amounts of PS-80.

| % w/w PS80 | | 0% | 0.27% | 0.67% | 1.33% | 2.00% | 2.67% |
|---|---|---|---|---|---|---|---|
| HP-SEC | % MPP | 99.3 | 99.3 | 99.5 | 99.5 | 99.5 | 99.4 |
| | % Agg | 0.1 | 0.1 | 0.2 | 0.2 | 0.1 | 0.1 |
| oVMD | d10 (μm) | 0.5 | 0.5 | 0.5 | 0.5 | 0.5 | 0.5 |
| | d50 (μm) | 1.8 | 1.8 | 1.8 | 1.7 | 1.2 | 1.5 |
| | d90 (μm) | 4 | 3.9 | 4.0 | 3.9 | 2.9 | 4.6 |
| | Span | 1.9 | 1.9 | 2.0 | 2.0 | 2.1 | 2.7 |
| Residual moisture (%) | | 1.6 | 1.6 | 1.7 | 1.9 | 1.9 | 0.4 |
| Tg | Open (° C.) | 123 | 124 | 121 | 121 | 121 | 124 |
| | Closed (° C.) | 99 | 98 | 96 | 95 | 96 | 115 |

| | | | | | | |
|---|---|---|---|---|---|---|
| cBD (g/cm3) | 0.58 | 0.60 | 0.59 | 0.59 | nm* | 0.55 |
| SSA (m2/g) | 4.60 | 5.02 | 4.37 | 4.05 | nm | 4.24 |

*nm-not measured

The analysis shows that powder properties are largely equivalent to the control formulation irrespective of % (w/w) amount of PS-80.

The formulations described in Table 22 were next analyzed for the content of sub-visible particles (SVPs). Sub-visible particles (SVP) counts were measured using the microflow imaging technology (MFI). MFI combines microfluidic flow microscopy and high resolution imaging particle analysis to quantify SVP counts and bin these counts across a particle size range. Prior to testing, powder samples were dissolved in water, and gently swirled to ensure uniform particle distribution then loaded on Protein Simple MFI 5200 (CA, USA). The results were reported as the counts for different particle sizes (<1 μm, <2 μm, <5 μm, <10 μm and <25 μm) per ml. FIG. 10A shows that inclusion of 0.27% (w/w/) PS-80 in the dry powder formulation reduces the absolute number of SVPs per ml on reconstitution. The reduction in SVPs counts decreases with increasing concentration of PS-80. Significant decreases in SVPs were seen on addition of 0.67% (w/w) PS-80, with a negligible amount of SVPs with a particle diameter of greater than 5 μm. The trend was observed when the formulation was reconstituted to a concentration of 30 mg/ml FAB₁ or 2.5 mg/ml FAB₁ (FIG. 10B).

Formulation characterization and analysis of SVPs were carried out as described above for a second excipient-containing formulation. In this study, poloxamer 188, as opposed to PS-80, was used as the excipient.

Multiple % w/w amounts of poloxamer 188 were examined. The formulation compositions and process parameters for the generation of each formulation batch were as described in Table 22 for PS-80-containing formulations. The amount of trehalose was modified to compensate for the variable amount of poloxamer 188.

Aggregate content, oVMD, residual moisture content, Tg, cBD and SSA were also measured using the methods described in the preceding examples. Results of the powder property analysis are shown in Table 25.

TABLE 25
Powder properties of dry powder formulations comprising
FAB₁ and varying (w/w) amounts of Poloxamer-188.

| %w/w P-188 | | 0 | 0.67% | 1% | 1.67% | 2.67% | 10% |
|---|---|---|---|---|---|---|---|
| HP-SEC | % MPP | 99.3 | 99.5 | 99.3 | 99.5 | 99.5 | 99.3 |
| | % Agg | 0.1 | 0.2 | 0.2 | 0.2 | 0.2 | 0.2 |
| oVMD | d10 (μm) | 0.5 | 0.5 | 0.5 | 0.5 | 0.5 | 0.5 |
| | d50 (μm) | 1.8 | 1.9 | 1.8 | 1.8 | 1.7 | 1.8 |
| | d90 (μm) | 4.0 | 4.2 | 3.9 | 4.2 | 4.1 | 4.3 |
| | Span | 1.9 | 2.0 | 1.9 | 2.1 | 2.1 | 2.1 |
| Residual moisture (%) | | 1.6 | 1.3 | 1.9 | 1.2 | 1.2 | 1.5 |
| Tg | Open (° C.) | 123 | 122 | 121 | 123 | 123 | 122 |
| | Closed (° C.) | 99 | 102 | 94 | 103 | 103 | 98 |

| | | | | | | |
|---|---|---|---|---|---|---|
| cBD (g/cm3) | 0.58 | 0.60 | 0.57 | 0.67 | 0.75 | nm* |
| SSA (m2/g) | 4.60 | nm* | 4.71 | nm* | 4.45 | nm* |

*nm-not measured

The aerosol properties of the Poloxamer-188 formulations were also analyzed using the methods disclosed in Example 7. The results are shown in Table 26.

TABLE 26
aerosol performance of formulations comprising
Poloxamer-188 (P188)

| | | FPM | |
|---|---|---|---|
| | % FPF | (<5.0 um) | MMAD |
| Description | (<5.0 um) | (mg) | (um) |
| 40% FAB₁, control | 78 | 5.5 | 2.55 |
| 40% FAB₁, | 67 | 4.5 | 2.61 |
| 0.67% w/w P188 | | | |
| 40% FAB₁, | 86 | 5.4 | 2.82 |
| 1% w/w P188 | | | |
| 40% FAB₁, | 66 | 4.2 | 2.76 |
| 1.67% w/w P188 | | | |
| 40% FAB₁, | 70 | 4.6 | 2.91 |
| 2.67% w/w P188 | | | |
| 40% FAB₁, | 56 | 3.0 | 3.41 |
| 10% w/w P188 | | | |

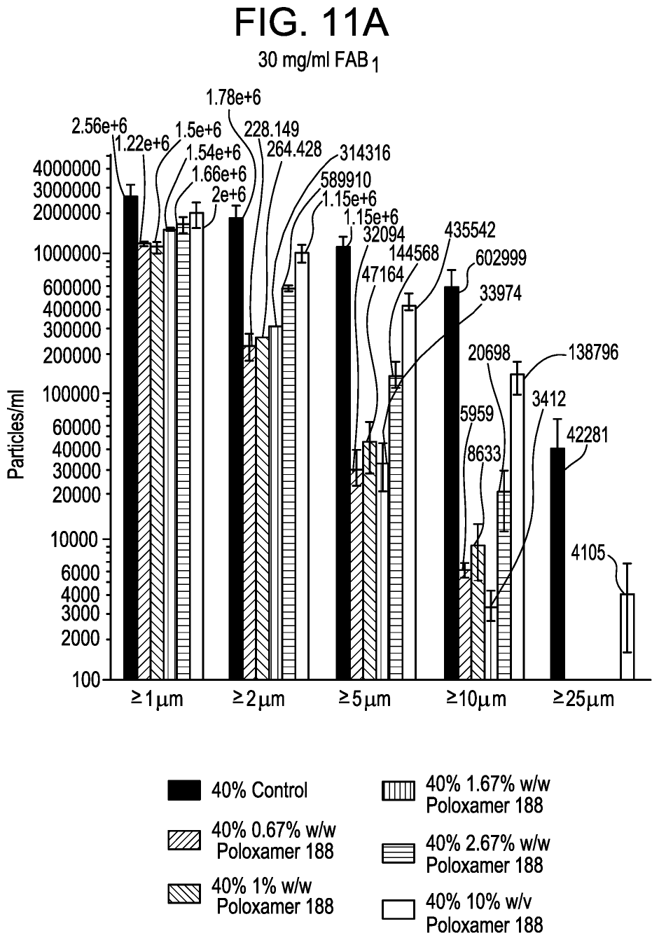

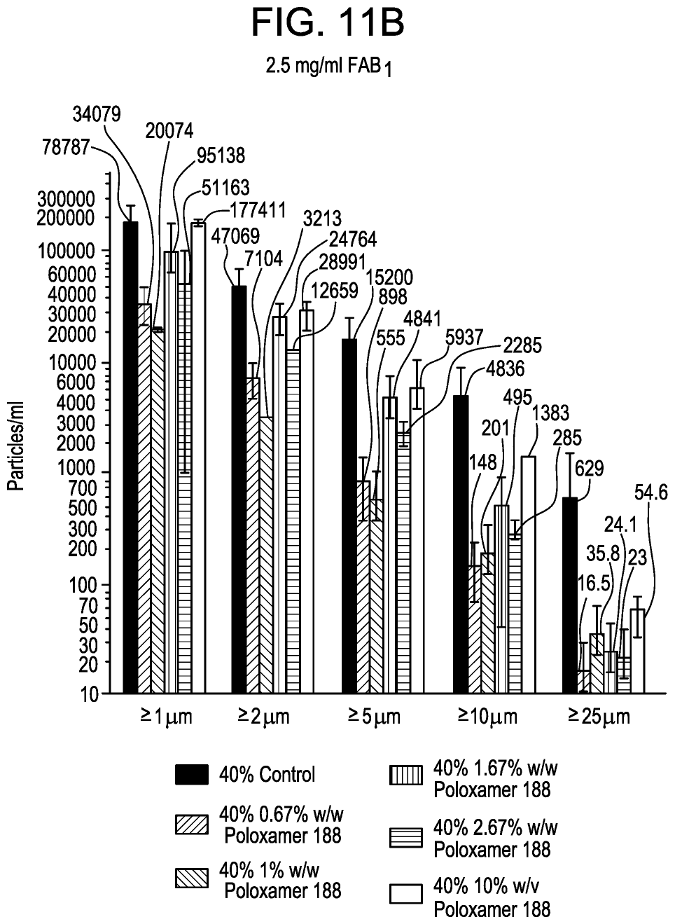

The P188 formulations were analyzed for SVP content using the methods described above. FIG. 11A shows that inclusion of 0.67% (w/w) P188 in the dry powder formulation reduces the absolute number of SVPs per ml on reconstitution. The trend was observed when the formulation was reconstituted to a concentration of 30 mg/ml FAB₁ (FIG. 11A) or 2.5 mg/ml FAB₁ (FIG. 11B).

### Example 9 Characterization of Leucine/Trileucine Formulations Comprising 1.1% (W/W) Ps-80

In this example the powder properties of a dry powder formulation comprising either 1% or 40% (w/w) Fab′ and 1.1% (w/w/) PS-80 were analysed. The complete formulation compositions are shown in Table 27. Formulations were manufactured as described in Example 6.

TABLE 27
By weight amounts of excipients in dry powder
formulations comprising 1.1% (w/w) PS-80 and
either 40% (w/w) or 1% (w/w) Fab₁.

| | Fab₁ 40% | Fab₁ 1% |
|---|---|---|
| | (w/w) | (w/w) |
| Trileucine | 2 | 2 |
| Leucine | 10.5 | 10.5 |
| Trehalose | 37.9 | 76.9 |
| Citrate buffer | 8.5 | 8.5 |

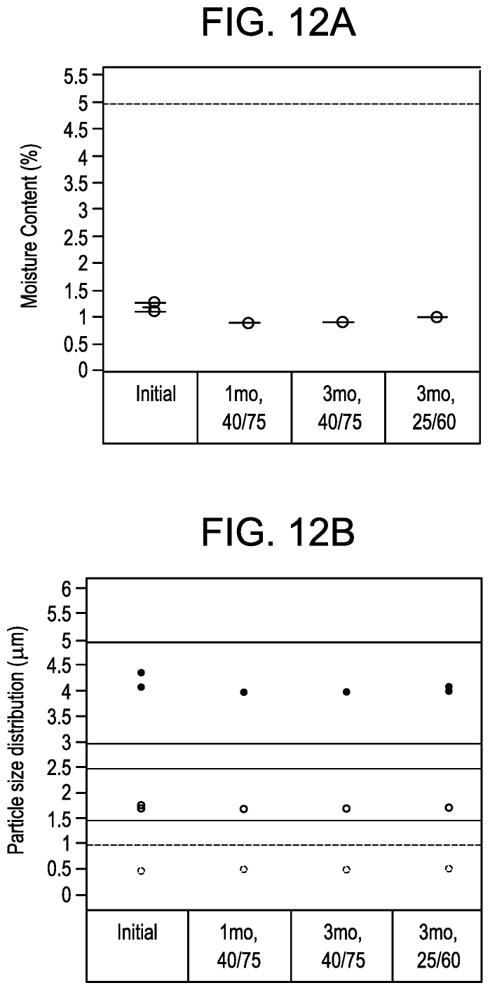

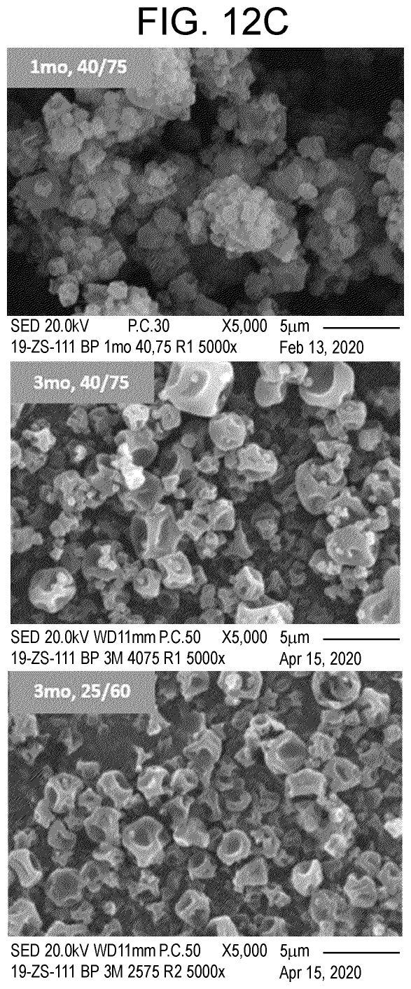

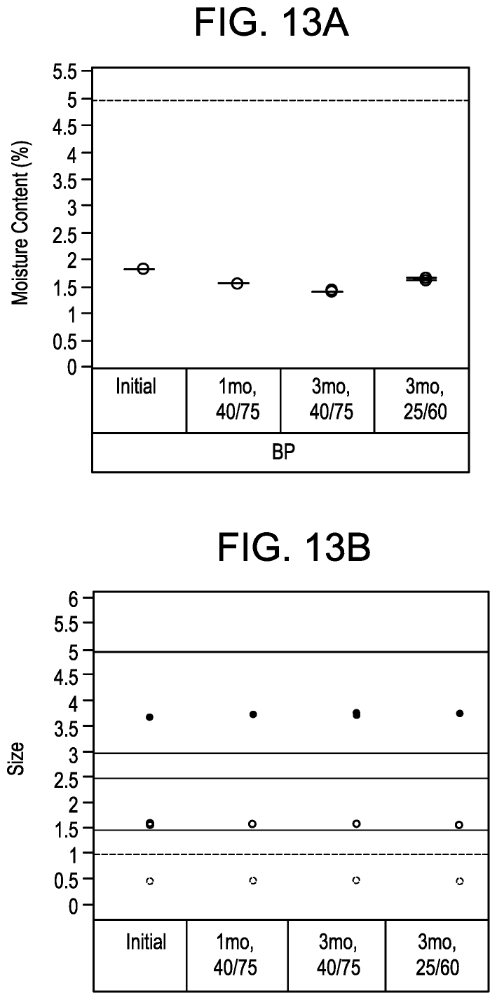

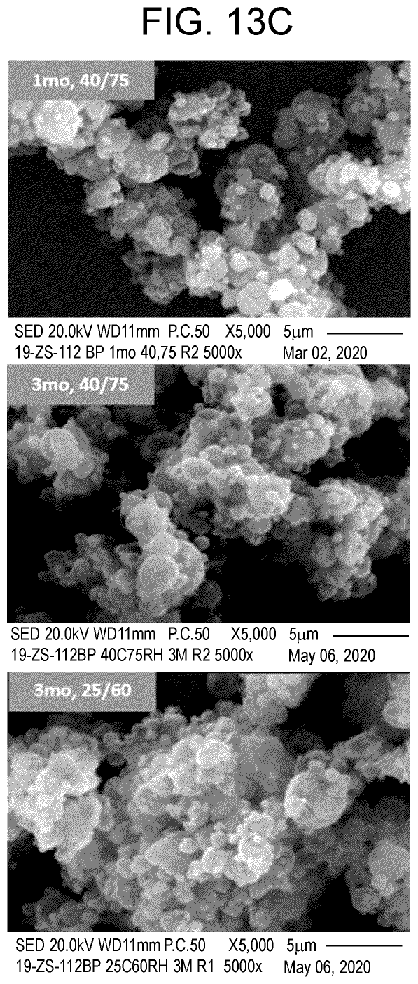

The stability of the formulations were analysed following storage for one or three months at either 40° C. and 75% relative humidity (40/75) or 25° C. and 60% relative humidity (25/60). Particle size distributions, moisture content and surface rugosity were tested. FIGS. 12A and B show that moisture content and particle size distributions remained stable over time for formulations comprising 40% (w/w) Fab₁. FIG. 12C shows that the morphology of the particles remains consistent over time. FIGS. 13A and 13B show that moisture content and particle size distributions remained stable over time for formulations comprising 1% (w/w) Fab₁. FIG. 13C shows that the morphology of the particles remains consistent over time.

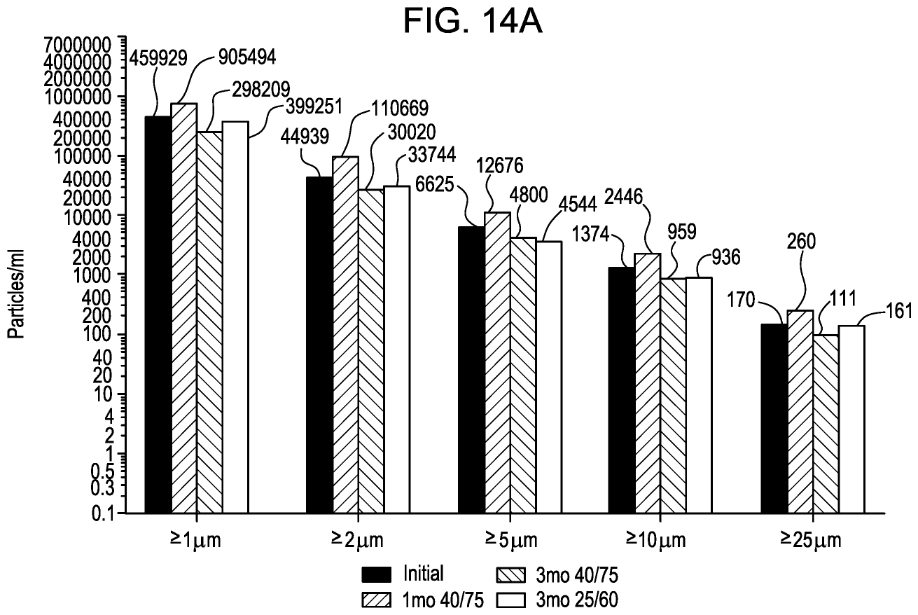

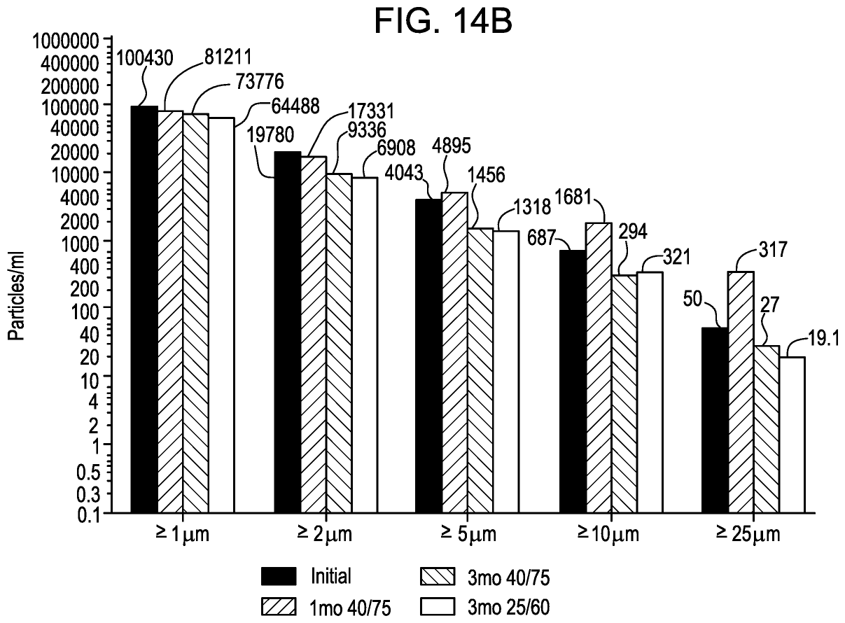

The formation of SVPs on reconstitution following storage at either 40/75 for 1 or 3 months, or 25/60 for 3 months was analysed. Analysis was carried out as described in Example 9. FIG. 14A shows that, on reconstitution of the 40% (w/w) Fab₁ formulation to a Fab₁ concentration of 30 mg.ml, the amount of SVPs forming under each condition is unchanged. FIG. 14B shows that, on reconstitution of the 1% (w/w) Fab₁ formulation to a Fab₁ concentration of 0.75 mg/ml, the amount of SVPs forming under each condition is unchanged.

Aerosol characteristics were also test following storage. The results are shown in Tables 28 and 29.

TABLE 28
Aerosol performance of formulations comprising 40% (w/w)
Fab₁ and 1.1% (w/w) PS-80 immediately following
manufacture and after storage for 1 or 3 months at 40/75
or 3 months at 25/60.

| | | 1 m | 3 m | 3 m |
|---|---|---|---|---|
| | T = 0 | 40/75 | 40/75 | 25/60 |
| % FPF (<5 um) | 81 | 87 | 77 | 78 |
| FPM (<5 um) (mg) | 5.2 | 5.1 | 4.7 | 5.1 |
| MMAD (μm) | 2.97 | 2.77 | 3.20 | 3.16 |
| DD (%) | 85 | 87 | 82 | 84 |

TABLE 29
Aerosol performance of formulations comprising 1% (w/w)
Fab₁ and 1.1% (w/w) PS-80 immediately following
manufacture and after storage for 1 or 3 months at 40/75
or 3 months at 25/60.

| | | 1 m | 3 m | 3 m |
|---|---|---|---|---|
| | T = 0 | 40/75 | 40/75 | 25/60 |
| % FPF (<5 um) | 79 | 79 | 73 | 73 |
| FPM (<5 um) (mg) | 0.12 | 0.11 | 0.11 | 0.1 |
| MMAD (μm) | 3.11 | 3.05 | 3.24 | 3.27 |
| DD (%) | 77 | 83 | 81 | 78 |

The percent delivered dose (DD) was also characterized following storage of each formulation under each condition. The results are shown in Tables 28 and 29.

The potency of Fab_{′} in each of the formulations described in Table 27 was also tested following storage at 40/75 for 1 or 3 months, or 25/60 for 3 months.

Potency was determined using homogeneous time resolved fluorescence (HTRF). HTRF combines fluorescence resonance energy transfer technology (FRET) with time-resolved measurements (TR). When two fluorophores, a donor and acceptor, are in close proximity to each other, excitation of the donor prompts an energy transfer to the acceptor, thus creating a FRET signal. In this assay, Streptavidin-Europium Cryptate, bound to biotinylated human TSLP, is the donor and a d2 labelled anti-TSLP mAb is the acceptor. FAB₁ binds to human TSLP and prevents the binding of the labelled mAb. This in turn increases the distance between the donor and acceptor fluorophores and results in a decrease in FRET signal.

After assessing parallelism between Reference Standard and assay control or between Reference Standard and test samples, a constrained four parameter logistic (4PL) curve fit is performed, and the relative potencies of FAB₁ assay control and test samples are calculated by dividing the IC50 value of the Reference Standard by the IC50 value of the assay control or each test sample and multiplying by 100%.

Potency levels of Fab₁ were between 85 to 110% of the potency of Fab₁ immediately reconstituted (i.e., t=0) from the equivalent formulation.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

While various embodiments have been described above, it should be understood that they have been presented only as illustrations and examples of the present technology, and not by way of limitation. It will be apparent to persons skilled in the relevant art that various changes in form and detail can be made therein without departing from the spirit and scope of the present technology. Thus, the breadth and scope of the present technology should not be limited by any of the above-described embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A dry powder formulation including a plurality of microparticles, the microparticles comprising:
a. leucine;
b. about 0.5% to about 10% trileucine by weight; and
c. an active agent,
wherein the leucine and the trileucine are present at a ratio of leucine:trileucine of about 0.1:1 to about 30:1 by weight.

2. The dry powder formulation of claim 1, wherein the dry powder formulation has a compressed bulk density of about 0.4 to about 1.0 g/cm³.

3. The dry powder formulation of any preceding claim, further comprising a glass stabilization agent.

4. The dry powder formulation of claim 3, wherein the glass stabilization agent is an amorphous saccharide or a buffer.

5. The dry powder formulation of claim 3, wherein the glass stabilization agent comprises an amorphous saccharide and a buffer.

6. The dry powder formulation of claim 4 or claim 5, wherein the amorphous saccharide is selected from the group consisting of trehalose, sucrose, raffinose, inulin, dextran, mannitol, and cyclodextrin.

7. The dry powder formulation of any one of claims 4-6, wherein the buffer is selected from the group consisting of a citrate buffer, a phosphate buffer, a histidine buffer, a glycine buffer, an acetate buffer and a tartrate buffer.

8. The dry powder formulation of any one of claims 4-7, wherein the amorphous saccharide is trehalose.

9. The dry powder formulation of any one of claims 1-8, wherein the active agent is an antibody or an antigen binding fragment thereof.

10. The dry powder formulation of any one of claims 1-9, wherein the ratio of leucine:trileucine is from about 1:1 to about 12:1 by weight, optionally from about 1:1 to about 7:1 by weight, optionally about 5.25:1 by weight.

11. The dry powder formulation of any one of claims 1-10, comprising about 8% to about 11% leucine by weight and about 2% to about 4% trileucine by weight.

12. The dry powder formulation of any one of claims 1-11, comprising about 10.5% leucine by weight and about 2% trileucine by weight.

13. The dry powder formulation of any one of claims 1-12, further comprising a surfactant, wherein the surfactant is optionally selected from polysorbate-20 (PS-20), polysorbate-40 (PS-40), polysorbate-60 (PS-60), polysorbate-80 (PS-80) and poloxamer-188.

14. The dry powder formulation of claim 13, wherein the surfactant is PS-80, wherein optionally PS-80 is present at a concentration in the range of from about 0.27% by weight to about 2.7% by weight, optionally from about 0.67% by weight to about 1.33% by weight.

15. The dry powder formulation of claim 14, wherein the PS-80 is present at a concentration of about 1.1% by weight.

16. The dry powder formulation of any one of claims 2-15, wherein the compressed bulk density is about 0.5 g/cm³ to about 0.8 g/cm³.

17. A dry powder formulation including a plurality of microparticles, the microparticles comprising about 10.5% leucine by weight, about 2% trileucine by weight, about 8.5% citrate buffer by weight, about 1% to about 40% active agent by weight, about 1.1% by weight polysorbate-80, and trehalose in an amount by weight amount to make up to 100%.

18. The dry powder formulation of claim 17, wherein the active agent is an antibody or an antigen binding fragment thereof.

19. A method of preparing a dry powder formulation, comprising:
a. preparing a liquid feedstock, comprising:
i. leucine;
ii. about 0.1 mg/mL to about 6 mg/mL trileucine;
iii. an active agent; and
iv. a liquid solvent;
wherein the leucine and the trileucine are present at a concentration ratio of leucine:trileucine of about 0.1:1 to about 30:1;
b. atomizing the liquid feedstock; and
c. drying the atomized liquid feedstock to form a plurality of microparticles.

20. The method of claim 19, wherein the liquid feedstock further comprises a glass stabilization agent, wherein optionally the glass stabilization agent comprises an amorphous saccharide and a buffer.

21. The method of claim 20, wherein the amorphous saccharide is selected from the group consisting of trehalose, sucrose, raffinose, inulin, dextran, mannitol, and cyclodextrin.

22. The method of either claim 20 or 21, wherein the buffer is selected from the group consisting of a citrate buffer, a phosphate buffer, a histidine buffer, a glycine buffer, an acetate buffer and a tartrate buffer.

23. The method of any one of claims 19-22 wherein the active agent is an antibody or an antigen binding fragment thereof.

24. The method of any one of claims 19-23, wherein step (a) further comprises adding a surfactant to the liquid feedstock, wherein the surfactant is optionally selected from polysorbate-20 (PS-20), polysorbate-40 (PS-40), polysorbate-60 (PS-60), polysorbate-80 (PS-80) and poloxamer-188.

25. The method of claim 24, wherein the surfactant is PS-80, wherein optionally the PS-80 is present in the liquid feedstock at a concentration in the range of from about 0.02% by weight to about 0.2% by weight.

26. The method of any one of claims 19-25, wherein the concentration ratio of leucine:trileucine in the feedstock is from about 1:1 to about 12:1, wherein optionally the concentration ratio of leucine:trileucine in the feedstock is about 5.25:1.

27. A method for preparing a dry powder formulation comprising a plurality of microparticles having a compressed bulk density of about 0.4 to about 1.0 g/cm³, the method comprising:
incorporating leucine and trileucine into the dry powder formulation at a ratio of leucine:trileucine of about 0.1:1 to about 30:1 by weight.

28. A method for preparing a dry powder formulation comprising a plurality of microparticles having a reduced number of sub-visible particles compared to a reference formulation, the method comprising incorporating:
i. leucine and trileucine into the dry powder formulation at a ratio of leucine:trileucine of about 0.1:1 to about 30:1 by weight;
ii. a surfactant, such as PS-80, into the dry powder formulation at a concentration in the range of from about 0.27% by weight to about 2.7% by weight.

29. The method of claim 28, wherein the reference formulation is equivalent to the formulation prepared by the method except it does not comprise the surfactant.

30. A method for delivery of a dry powder formulation to the lungs of a mammalian patient, the method comprising administering to the mammalian patient by inhalation the dry powder formulation of any one of claims 1-18, in an aerosol form.

31. A method for treating a medical condition in a mammalian patient, comprising administering to the mammalian patient by inhalation the dry powder formulation of any one of claims 1-18, in an aerosol form.

32. The method of claim 31, wherein the dry powder formulation is administered by a dry-powder inhaler (DPI).

33. The formulation of any one of claims 1-18 for use in a method of treatment, wherein the formulation is to be administered by inhalation.


SEQUENCE LISTING (10 sequences)

SEQ ID NO: 1 - protein, 5 aa, Homo sapiens
  MISC_FEATURE: HCDR1 FAB1
TYGMH

SEQ ID NO: 2 - protein, 17 aa, Homo sapiens
  MISC_FEATURE: HCDR2 FAB1
VIWYDGSNKHYADSVKG

SEQ ID NO: 3 - protein, 13 aa, Homo sapiens
  MISC_FEATURE: HCDR3 FAB1
APQWELVHEAFDI

SEQ ID NO: 4 - protein, 122 aa, Homo sapiens
  MISC_FEATURE: HEAVY CHAIN VH FAB1
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSS

SEQ ID NO: 5 - protein, 11 aa, Homo sapiens
  MISC_FEATURE: LCDR1 FAB1
GGNNLGSKSVH

SEQ ID NO: 6 - protein, 7 aa, Homo sapiens
  MISC_FEATURE: LCDR2 FAB1
DDSDRPS

SEQ ID NO: 7 - protein, 11 aa, Homo sapiens
  MISC_FEATURE: LCDR3 FAB1
QVWDSSSDHVV

SEQ ID NO: 8 - protein, 108 aa, Homo sapiens
  MISC_FEATURE: LIGHT CHAIN VL
  MISC_FEATURE: LIGHT CHAIN VL FAB1
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVL

SEQ ID NO: 9 - DNA, 366 bp, Homo sapiens
  misc_feature: VARIABLE HEAVY CHAIN FAB1
cagatgcagttggttgaatctggtggcggcgtggtgcagcctggcagatctctgagactgtcttgtgccgcctccggcttcaccttcagaacctacggaatgcactgggtccgacaggcccctggcaaaggattggaatgggtcgccgtgatttggtacgacggctccaacaagcactacgccgactccgtgaagggcagattcaccatcaccagagacaactccaagaacaccctgaacctgcagatgaactccctgagagccgaggacaccgccgtgtactattgtgctagagcccctcagtgggaactcgtgcatgaggcctttgacatctggggccagggaacaatggtcaccgtctcctca

SEQ ID NO: 10 - DNA, 324 bp, Homo sapiens
  misc_feature: FAB1 VARIABLE LIGHT CHAIN
tcatatgttcttacacaaccaccgtcggtttcggttgctccaggacaaacagctcgaattacatgcggaggaaacaacctcggatcgaagtcggttcactggtatcaacaaaagccaggacaagctccagttctcgtggtgtacgatgattcagatcgaccatcatggatcccagagcgattctcaggatcaaactcgggaaatactgccacgctcacaatttcacgcggagaagcgggagatgaagctgattactattgccaagtgtgggactcgtcgtcagatcatgttgttttcggaggtggaacaaagctcacagtgctc